(19) United States
(12) Patent Application Publication
(10) Pub. No.: US 2022/0096652 A1
(43) Pub. Date: Mar. 31, 2022

(54) ACETAL-BASED CLEAVABLE LINKERS

(71) Applicant: Synaffix B.V., Oss (NL)

(72) Inventors: Jorge Merijn Mathieu VERKADE, Eindhoven (NL); Arnoldus Jacobus VAN SCHAIK, Oss (NL); Remon VAN GEEL, Lithoijen (NL); Maria Antonia WIJDEVEN, Oss (NL); Sander Sebastiaan VAN BERKEL, Wijchen (NL); Floris Louis VAN DELFT, Nijmegen (NL)

(73) Assignee: Synaffix B.V., Oss (NL)

(21) Appl. No.: 17/539,929

(22) Filed: Dec. 1, 2021

Related U.S. Application Data
(63) Continuation of application No. PCT/EP2020/065395, filed on Jun. 3, 2020.

(30) Foreign Application Priority Data
Jun. 3, 2019 (EP) 19177933.9

(51) Int. Cl. A61K 47/68; A61K 31/4995

(52) U.S. Cl. CPC A61K 47/6851; A61K 47/6803; A61K 31/4995

(57) ABSTRACT

The current invention concerns compounds, such as antibody-conjugates, with an enhanced selectivity of payload release inside a tumour or in the tumour microenvironment versus payload release in circulation or in healthy cells. The enhanced selectivity is achieved by incorporation of a cleavable linker that requires two consecutive mechanisms for release of the payload. The compounds according to the invention have structure (1):

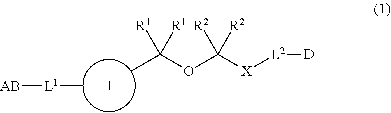

or a salt thereof, wherein AB is an antibody or a reactive moiety capable of reacting with a functional group on an antibody, L¹ and L² are linkers, moiety I contains an activating group and an aromatic ring, X is O or N and D is the payload. The invention further concerns application of these compounds in for example a method of targeting a cell and a method for enhancing the bystander effect of an amino-containing payload. The invention also concerns the payloads that may be released from the compounds according to the invention.

### CROSS-REFERENCE TO RELATED APPLICATIONS

The present application is a Continuation of International Patent Application No. PCT/EP2020/065395, filed Jun. 3, 2020, which claims priority to European Patent Application No. 19177933.9 filed Jun. 3, 2019; the entire contents of all of which are hereby incorporated by reference.

### FIELD OF THE INVENTION

The present invention relates to conjugates of payloads containing an amine moiety. More specifically, the invention relates to conjugates, compositions and methods that are amenable to (enzymatic, acidic) bioactivation and cleavage. Such enzyme-activatable conjugates compounds, compositions, and methods can be useful, for example, in providing novel prodrugs for targeted delivery of payloads, such as potent therapeutics.

### BACKGROUND OF THE INVENTION

Antibody-drug conjugates (ADC), considered as magic bullets in therapy, are comprised of an antibody to which is attached a pharmaceutical agent. The antibodies (also known as ligands) can be small protein formats (scFv's, Fab fragments, DARPins, Affibodies, etc.) but are generally monoclonal antibodies (mAbs) which have been selected based on their high selectivity and affinity for a given antigen, their long circulating half-lives, and little to no immunogenicity. Thus, mAbs as protein ligands for a carefully selected biological receptor provide an ideal delivery platform for selective targeting of pharmaceutical drugs. For example, a monoclonal antibody known to bind selectively with a specific cancer-associated antigen can be used for delivery of a cytotoxic agent to the tumour, via binding, internalization, intracellular processing and finally release of active catabolite. As a result, the tumour cells can be selectively eradicated, while sparing normal cells which have not been targeted by the antibody. Similarly, conjugation of an antibacterial drug to an antibody can be applied for treatment of bacterial infections, conjugates of anti-inflammatory drugs are under investigation for the treatment of autoimmune diseases and attachment of an oligonucleotide to an antibody is a potential promising approach for the treatment of neuromuscular diseases. There, the concept of targeted delivery of active pharmaceutical drug to a specific cellular location of choice is a powerful approach for the treatment of a wide range of diseases, with many beneficial aspects versus systemic delivery of the same drug.

A chemical linker is employed to attach a pharmaceutical drug to an antibody. This linker needs to possess a number of key attributes, including the requirement to be stable in plasma after drug administration for an extended period of time. A stable linker enables localization of the ADC to the projected site or cells in the body and prevents premature release of the payload in circulation, which would indiscriminately induce undesired biological response of all kinds, thereby lowering the therapeutic index of the ADC. Upon internalization, the ADC should be processed such that the payload is effectively released so it can bind to its target.

There are two families of linkers, non-cleavable and cleavable. Non-cleavable linkers consist of a chain of atoms between the antibody and the payload, which is fully stable under physiological conditions, irrespective of which organ or biological compartment the antibody-drug conjugate resides in. As a consequence, liberation of the payload from an ADC with a non-cleavable linker relies on the complete (lysosomal) degradation of the antibody after internalization of the ADC into a cell. As a consequence of this degradation, the payload will be released, still carrying the linker, as well as a peptide fragment and/or the amino acid from the antibody the linker was originally attached to. Cleavable linkers utilize an inherent property of a cell or a cellular compartment for selective release of the payload from the ADC, which generally leaves no trace of linker after metabolic processing. For cleavable linkers, there are three commonly used mechanisms: 1) susceptibility to specific enzymes, 2) pH-sensitivity, and 3) sensitivity to redox state of a cell (or its microenvironment).

Enzyme-based strategies are generally based on the endogenous presence of specific proteases, esterases, glycosidases or others. For example, the majority of ADCs used in oncology utilize the dominant proteases found in a tumour cell lysosome for recognition and cleavage of a specific peptide sequence in the linker. Dubowchik et al., Bioconjug Chem. 2002, 13, 855-69, incorporated by reference, pioneered the discovery of specific dipeptides as an intracellular cleavage mechanism by cathepsins. Other enzymes that are known to be upregulated in the tumour lysozyme or the tumour microenvironment are plasmin, matrix metallo proteases (MMPs), urokinase, and others, all of which may recognize a specific peptide sequence in the ADCs and induce release of payload from the linker by hydrolytic cleavage of one of the peptide bonds. Esterases may also be employed for intracellular release of payload upon hydrolysis of an ester bond, for example it was demonstrated by Barthel et al, J. Med. Chem. 2012, 55, 6595-6607, incorporated by reference, that human carboxylesterase 2 (CES2, hiCE) demonstrated an in vivo antitumor efficacy of a doxorubicin prodrug against CES2-positive xenografts that was better than or equal to that of payload itself. Thirdly, various glycosidases may be employed for selective cleavage of a specific monosaccharide, in particular galactosidase (for removal of galactose) or glucuronidase (for removal of glucuronic acid), as for example illustrated in respectively Torgov et al, Bioconj. Chem. 2005, 16, 717-721 and Jeffrey et al, J. Med. Chem. 2006, 17, 831-840, incorporated by reference. Other endogenous enzymes that may be employed for tumour-specific hydrolytic cleavage of bonds are for example phosphatases or sulfatases.

Besides the use of endogenous enzymes, local concentration enhancement of any enzyme of choice, which may not be naturally abundant, can be achieved by strategies such as systemic administration by intravenous injection, by intratumoural injection or by other methods such as ADEPT (antibody-directed enzyme prodrug therapy).

The acid-sensitivity strategy takes advantage of the lower pH in the endosomal (pH 5-6) and lysosomal (pH 4.8) compartments, as compared to the cytosol of a human cell (pH 7.4), to trigger hydrolysis of an acid labile group within the linker, such as a hydrazone, see for example Ritchie et al, mAbs 2013, 5, 13-21, incorporated by reference. Alternative acid-sensitive linker may also be employed, as for example based on silyl ethers, disclosed in US20180200273.

A third release strategy based on redox mechanisms exploits the higher concentrations of intracellularglutathione than in the plasma. Thus, linkers containing a disulfide bridge release a free thiol group upon reduction by glutathione, which may remain part of the payload or further self-immolate to release the free payload. Alternative reduction mechanisms for release of free payload can be based on the conversion of an (aromatic) nitro group or a (aromatic) azido group into an aniline, which may be part of a payload or part of a self-immolative assembly unit.

A self-immolative assembly unit in an antibody-drug conjugate links a drug unit to the remainder of the conjugate or its drug-linker intermediate. The main function of the self-immolative assembly unit is to conditionally release free drug at the site targeted by the ligand unit. The activatable self-immolative moiety comprises an activatable group and a self-immolative spacer unit. Upon activation of the activatable group, for example by enzymatic conversion of an amide group to an amino group or by reduction of a disulfide to a free thiol group, a self-immolative reaction sequence is initiated that leads to release of free drug by one or more of various mechanisms, which may involve (temporary) 1,6-elimination of a p-aminobenzyl group to a p-quinone methide, optionally with release of carbon dioxide and/or followed by a second cyclization release mechanism. The self-immolative assembly unit can part of the chemical spacer connecting the antibody and the payload (via the functional group). Alternatively, the self-immolative group is not an inherent part of the chemical spacer, but branches off from the chemical spacer connecting the antibody and the payload.

The majority of antibody-drug conjugates that have been market-approved or are currently in late-stage clinical trials employ one of the mechanisms described above for release of active drug. For example, Adcetris® is an ADC used for treatment of various hematological tumours and is comprised of a CD30-targeting antibody (ligand), connected to a highly potent tubulin inhibitor MMAE (payload) via a linker that consists of a cathepsin-sensitive fragment connected to a self-immolative p-aminobenzyloxycarbonyl group (PAB). Other ADCs in pivotal trials that employ protease/peptidase-sensitive linkers are polatuzumab-vedotin, SYD985, ABT-414, Rova-T, ASG-22CE and DS-8201a. Protease-mediated release of payload is also part of the design of RG7861 (DSTA4637S), which is an ADC under development in an area outside oncology, specifically for treatment of bacterial infections.

Two ADCs have been approved (Besponsa® and Mylotarg®) that consist of an antibody connected to a DNA-damaging payload (calicheamicin) via an acid-sensitive group, in particular a hydrazone group. Similarly, sacituzumab govetican, an ADC in phase III clinical studies, employs release of payload via acidic hydrolysis of a carbonate group. A glutathione-sensitive disulfide group is part of the linker in mirvetuximab soravtansine to connect antibody to the maytansinoid payload DM4. Currently, more than 75 ADCs are in various stages of clinical trials, the at least 70% of which contain one form of a cleavable linker.

As described above, a self-immolative unit is part of the linker in many ADCs, which in most cases at least exists of an (acylated) para-aminobenzyl unit connected to a protease-sensitive peptide fragment for enzymatic release of the amino group. Besides the aminobenzyl group, other aromatic moieties may also be employed as part for the self-immolative unit, for example heteroaromatic moieties such as pyridine or thiazoles, see for example U.S. Pat. No. 7,754,681 and US2005/0256030. Substitution of the aminobenzyl group may be in the para position or in the ortho position, in both cases leading the same 1,6-elimination mechanism. The benzylic position may be substituted with alkyl or carbonyl derivatives, for example esters or amides derived from mandelic acid, as for example disclosed in WO2015/038426, incorporated by reference. The benzylic position of the self-immolative unit is connected to a heteroatom leaving group, typically based on, but not limited to, oxygen or nitrogen. Predominantly, the benzylic functional group exists of a carbamate moiety, which will release carbon dioxide upon triggering of the 1,6-elimination mechanism, and a primary or secondary amino group. The primary or secondary amino group may be part of the toxic payload itself, and may be an aromatic amino group or an aliphatic amino group. In the latter case, the amino group of the liberated payload will most likely have a pKa higher than and therefore be mostly in a protonated state at physiological conditions (pH 7-7.5), and specifically in the acidic environment of the tumour (pH<7).

The primary or secondary amino group may also be part of another self-immolative group, for example an N,N-dialkylethylenediamine moiety. The N,N-dialkylethylenediamine moiety at the other may be connected to another carbamate group to liberate, upon cyclization, an alcohol group as part of the toxic payload, as for example demonstrated by Elgersma et al, Mol. Pharm. 2015, 12, 1813-1835, incorporated by reference. The primary or secondary amino group of the carbamate moiety may also form part of an N,O-acetal, a method which has been used in several drug delivery strategies, for example to release 5-fluorouracil (Madec-Lougerstay et al, J. Chem. Soc. Perkin Trans I, 1999, 1369-1375) and SN-38 (Santi et al, J. Med. Chem. 2014, 57, 2303-2314). Most recently, a similar configuration was employed by Kolakowski et al, Angew. Chem. Int. Ed. 2016, 55, 7948-7951, incorporated by reference, for design of linkers with prolonged serum exposure because of the long circulation time of ADCs, in combination with a beta-glucuronidase-promoted release mechanism, to release aliphatic alcohols. The functional group at the benzylic position of the self-immolative aromatic moiety may also be a phenolic oxygen, see for example Toki et al, J. Org. Chem. 2002, 67, 1866-1872 and U.S. Pat. No. 7,553,816, incorporated by reference, however not an aliphatic alcohol because an aliphatic alcohol does not possess sufficient leaving group capacity (typical pKa 13-15). Another option for the benzylic functional group is a quaternary ammonium group, which will release a trialkylamino group or a heteroaryl amine upon elimination, as reported by Burke et al, Mol. Cancer Ther. 2016, 15, 938-945 and Staben et al, Nat. Chem. 2016, 8, 1112-1119, incorporated by reference.

Currently, payloads utilized in ADCs primarily include microtubule-disrupting agents [e.g. monomethyl auristatin E (MMAE) and maytansinoid-derived DM1 and DM4], DNA-damaging agents [e.g., calicheamicin, pyrrolobenzodiazepines (PBD) dimers, indolinobenzodiapines dimers, duocarmycins, anthracyclins], topoisomerase inhibitors [e.g. SN-38, exatecan and derivatives thereof, simmitecan] or RNA polymerase II inhibitors [e.g. amanitin]. Although ADCs have demonstrated clinical and preclinical activity, it has been unclear what factors determine such potency in addition to antigen expression on targeted tumour cells. For example, drug:antibody ratio (DAR), ADC-binding affinity, potency of the payload, receptor expression level, internalization rate, trafficking, multiple drug resistance (MDR) status, and other factors have all been implicated to influence the outcome of ADC treatment in vitro. In addition to the direct killing of antigen-positive tumour cells, ADCs also have the capacity to kill adjacent antigen-negative tumour cells: the so-called “bystander killing” effect, as originally reported by Sahin et al, Cancer Res. 1990, 50, 6944-6948 and for example studied by Li et al, Cancer Res. 2016, 76, 2710-2719. Generally spoken, cytotoxic payload that are neutral will show bystander killing whereas ionic (charged) payloads do not, as a consequence of the fact that ionic species do not readily pass a cellular membrane by passive diffusion. For example, evaluation of a range of exatecan derivatives indicated that acylation of the primary amine with hydroxyacetic acid provided a derivative (DXd) with substantially enhanced bystander killer versus various aminoacylated exatecan derivatives, as disclosed by Ogitani et al, Cancer Sci. 2016, 107, 1039-1046, incorporated by reference.

A disadvantage of the majority of the clinically tested and marketed ADCs in the field is that the toxic payload may induce dose-limiting off-target toxicities, reviewed by Donaghy et al, MAbs 2016, 8, 659-71, incorporated by reference. It was for example demonstrated by Thon et al. Blood 2012, 120, 1975-84, incorporated by reference, that ADCs can be taken up by differentiating hematopoietic stem cells, leading to release of toxic payload, inhibition of megakaryocyte proliferation and differentiation, thus preventing the generation of thrombocytes and finally resulting in thrombocytopenia. Similarly, it is believed that hydrazone linker instability played a role in the safety issues of Mylotarg®, which was withdrawn from the market in 2010 (but later re-introduced). It has been shown that linkers designed for proteolytic cleavage by cathepsins can also be cleaved by other enzymes like esterase Ces1c (reported by Dorywalska et al, Mol. Cancer Ther. 2016, 15, 958-970, incorporated by reference). In fact, it was demonstrated by Caculitan et al, Cancer Res. 2017, 7027-7037, incorporated by reference, that even in the absence of cathepsin B, peptide-based cleavable linkers readily undergo cellular processing to release free payload. Moreover, it was demonstrated by Zhao et al. (Mol. Cancer Ther. 2017, 16, 1866-1876, incorporated by reference) that excretion of elastase by differentiating neutrophils may cause premature release of toxic payload, and is one of the causes of neutropenia, a common adverse event in cancer patients treated with MMAE-based ADCs. Hence, there is a clear unmet need in the field to develop novel linkers with more selective cleavage and release of payload in the tumour microenvironment.

### SUMMARY OF THE INVENTION

The current invention is centred around antibody-drug conjugates with an enhanced selectivity of payload release inside a tumour or in the tumour microenvironment versus payload release in circulation or in healthy cells. The inventors have developed conjugates having an improved release process with enhanced selectivity of release in or near tumour cells. The enhanced selectivity is achieved by incorporation of a cleavable linker that requires two consecutive mechanisms for release of the (toxic) payload, both of which designed for enhanced rate in the tumour environment and with the second mechanism conditional to completion of the first mechanism. Specifically, the linker according to the invention contains a substituted benzylic O,O-acetal or O,N-acetal, that is first activated by an enzymatic hydrolysis or reduction mechanism (step 1), that induces enhanced acid-sensitivity and thus acid-mediated hydrolysis of the O,O-acetal or O,N-acetal (step 2) to release eventually an aliphatic alcohol or amino group, which is part of the payload. The mechanism of the release process is depicted in FIGS. 1A and 1B. A 1,6-elimination mechanism after step 1 is thereby disfavoured due to poor leaving group capacity of an aliphatic alcohol. A second advantage of the invention is that it allows for straightforward conversion of any amino-functionalized payload by a simple acylation step with the cleavable linker, which will liberate a hydroxy-acylated version of the original payload with enhanced bystander function.

The present invention thus concerns a compound according to formula (1):

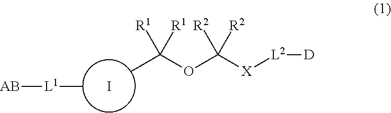

or a salt thereof, wherein
AB is an antibody or a reactive moiety capable of reacting with a functional group on an antibody;
L¹ is a linker;
moiety I is according to formula (2a), (2b), (2c) or (2d):

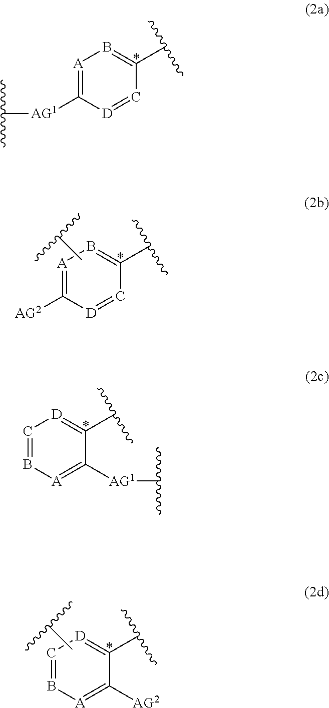

wherein
   * indicates the carbon atom connected to C(R¹)₂;
   each of A, B, C and D is independently selected from N and CR⁵, wherein R⁵ is H, optionally substituted alkyl, alkoxy, sulfonate, alkylamino or halogen wherein the alkyl may be interrupted by one or more heteroatoms, or in case of formula (2b) and (2d) at least one of A, B, C and D is CR⁵, wherein R⁵ is L¹-AB;
   AG¹ is an activating group selected from —S—S— and —NH-peptide-, wherein the peptide contains 1-10 amino acids;
   AG² is an activating group selected from the group consisting of —S—S—R¹⁰ and —NH-peptide-R¹¹, —NO₂, —N₃, —OR⁴ and —OC(O)R⁸, wherein the peptide contains 1-10 amino acids, wherein R⁴ is selected from phosphate, sulfate and monosaccharide, R⁸ is a C₂-C₁₂ alkyl group, R¹⁰ is an optionally substituted alkyl or aryl group; and R¹¹ is the optionally protected end group of the peptide;
each R¹ is independently selected from the group consisting of hydrogen, optionally substituted alkyl, optionally substituted aryl, C(O)OR⁶ and C(O)N(R⁶)₂;
each R² is independently selected from the group consisting of hydrogen, optionally substituted alkyl and optionally substituted aryl, or both occurrences of R² are joined together in a cyclic structure;
X is selected from O and NR³, wherein R³ is selected from the group consisting of H, alkyl, aryl, —C(O)-alkyl, —C(O)-aryl, —C(O)—O-alkyl, —C(O)—O-aryl, —C(O)—N(R⁶)₂, —C(O)—N(R⁶)₂, —S(O₂)—O-alkyl, S(O₂)—O-aryl, —S(O₂)—N(R⁶)₂ and —S(O₂)—N(R⁶)₂;
each R⁶ is independently selected from the group consisting of hydrogen, optionally substituted alkyl and optionally substituted aryl, or both occurrences of R⁶ on the same nitrogen atom are joined into in a cyclic structure;
L² is —C(O)-L³-C(O)— or -L³-C(O)—, wherein L³ is optionally substituted alkylene and/or optionally substituted arylene, wherein the alkylene may optionally be interrupted with 1-3, preferably at most 1, heteroatoms selected from O, NR⁷ and S(O)_{y}, wherein y=0, 1 or 2;
D is a payload.

The present invention further concerns a method of targeting a cell, comprising contacting the cell with a compound according to the invention, wherein AB is an antibody that specifically targets the cell or a receptor expressed on the cell.

The present invention further concerns a method of providing a payload to a cell, comprising contacting the cell with a compound according to the invention, wherein AB is an antibody that specifically targets the cell or a receptor expressed on the cell, wherein a payload having formula HX-L²-D is released near or in the cell.

The present invention further concerns a method of enhancing the bystander effect of an amino-containing payload by conversion into an hydroxyl-containing payload HO-L²-D, comprising contacting a cell with a compound according to the invention, wherein X=O and AB is an antibody that specifically targets the cell or a receptor expressed on the cell, wherein a payload having formula HO-L²-D is released near or in the cell.

The present invention further concerns a compound, which is the payload that may be released from the conjugate according to the invention, having a structure selected from the group consisting of (5a)-(5i):

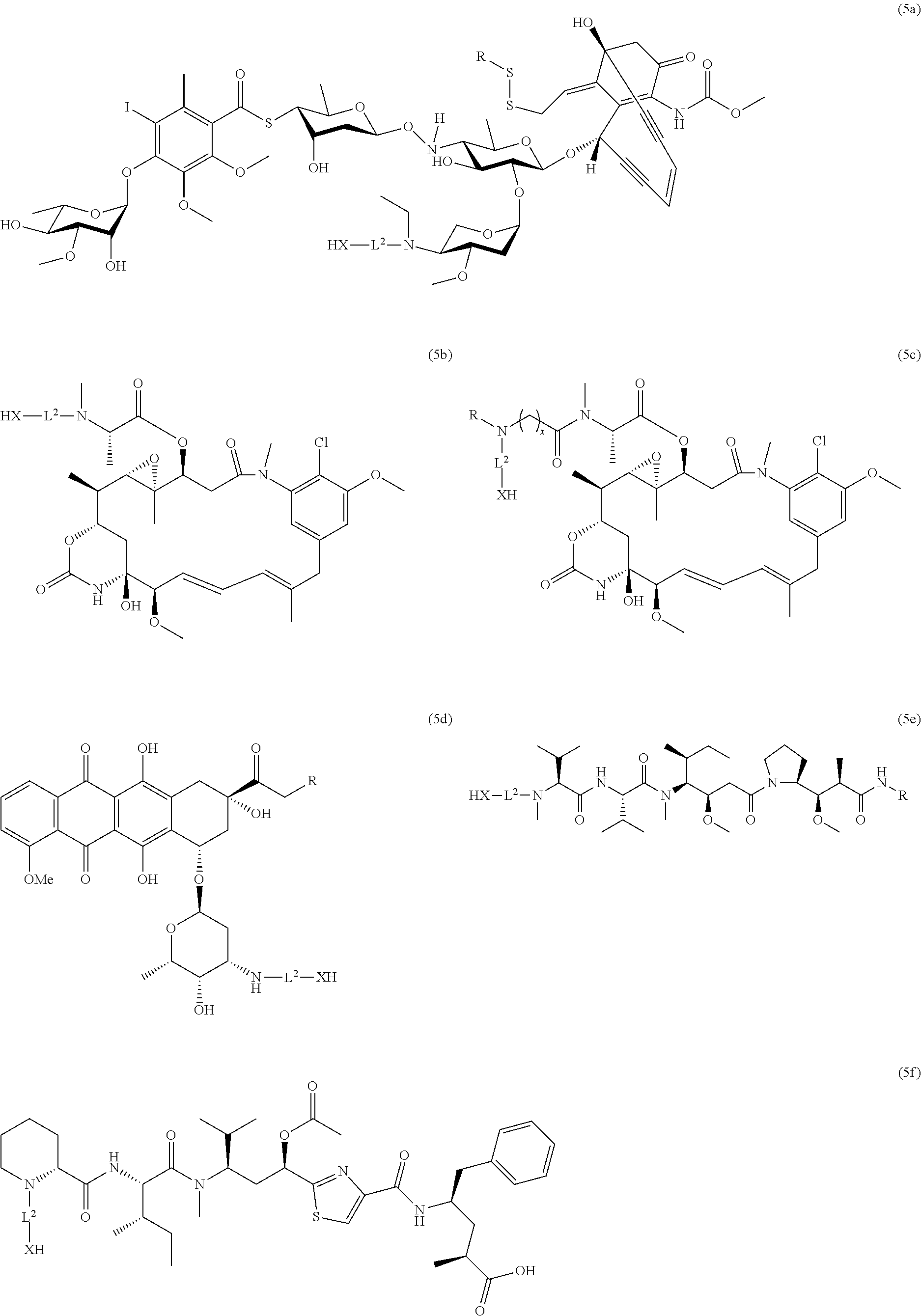

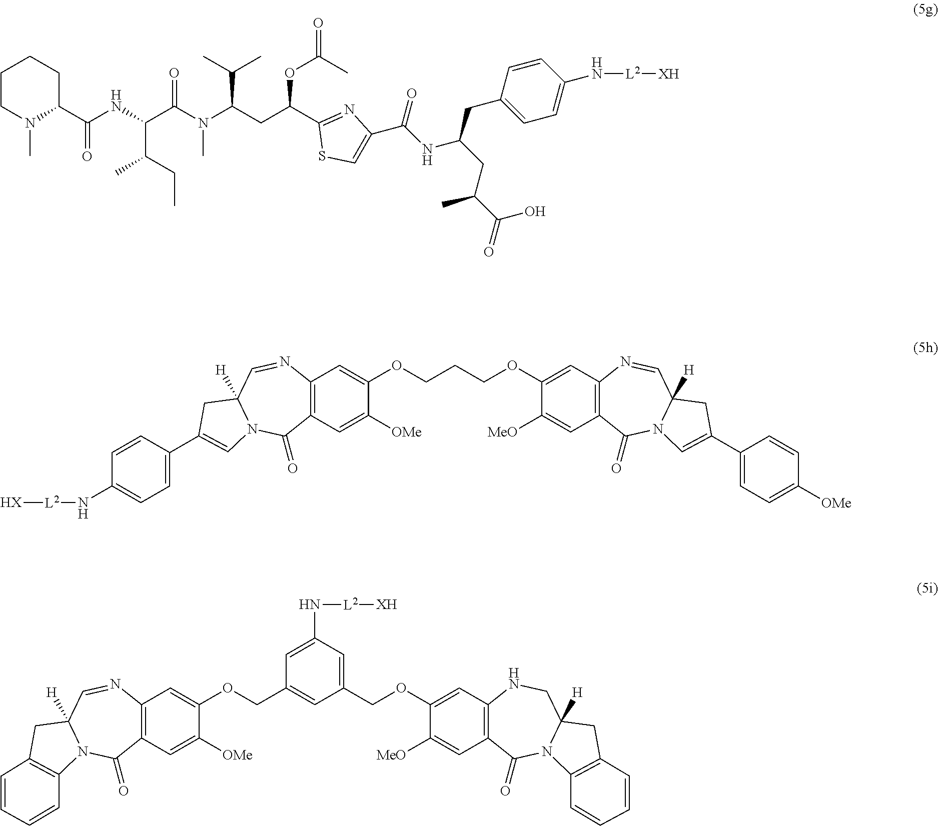

or a salt thereof, wherein:
X is selected from O and NR³, wherein R³ is selected from the group consisting of H, alkyl, aryl, —C(O)-alkyl, —C(O)-aryl, —C(O)—O-alkyl, —C(O)—O-aryl, —C(O)—N(R⁶)₂, —C(O)—N(R⁶)₂, —S(O₂)—O-alkyl, S(O₂)—O-aryl, —S(O₂)—N(R⁶)₂ and —S(O₂)—N(R⁶)₂;
L² is —C(O)-L³-C(O)— or -L³-C(O)—, wherein L³ is optionally substituted alkylene and/or optionally substituted arylene, wherein the alkylene or arylene is optionally substituted and the alkylene may optionally be interrupted with 1-3 heteroatoms selected from O and NR⁷, wherein R⁷ is selected from H and C₁-C₄ alkyl,

and wherein:
in structure (5a), R is —S(C₁-C₁₀ alkyl) or —C(R¹⁶)₂R¹⁷, wherein each R¹⁶ is independently selected from H or optionally substituted C₁-C₆ alkyl and R¹⁷ is selected from H, C₁-C₁₂ alkyl, -L⁶-OR¹⁸, (CH₂)ₛO-L⁶-OR¹⁸ or (CH₂)ₛC(O)NR¹⁹-L⁶-OR¹⁸, wherein L⁶ is a polar linker having 1-100 optionally substituted backbone atoms selected from C, N, O and S, R¹⁸ is H or methyl, s=1, 2 or 3, and R¹⁹ is selected from H and -L⁶-OR¹⁸;
in structure (5c), R is H or C₁-C₁₂ alkyl and x is an integer in the range 1-10;
in structure (5d), R is H or OH; and
in structure (5e), R is CH(Me)-CH(OH)-Ph.

### DESCRIPTION OF THE DRAWINGS

FIGS. 1A and 1B display the release of a payload with a hydroxy group from linkers based on a dual mechanism. In FIG. 1A it is shown how an acyl substituent on a p-aminobenzyloxy group deactivates protonation of the benzylic oxygen by an electron-withdrawing effect, while proteolytic removal of peptide leads to enhanced protonation and subsequent accelerated elimination of hydroxylated payload via a protonated p-quinone methide and 1,2-elimination. FIG. 1B shows how a similar mechanism is operative for a p¬-disulfide substituted benzylic system: upon reduction a p-mercaptobenzyloxy derivative is generated, which will accelerate benzylic protonation, in particular as its thiolate derivative (formed by deprotonation under physiological conditions). Similar mechanisms will pertain to a p-substituted benzylic O,N-acetal (not displayed).

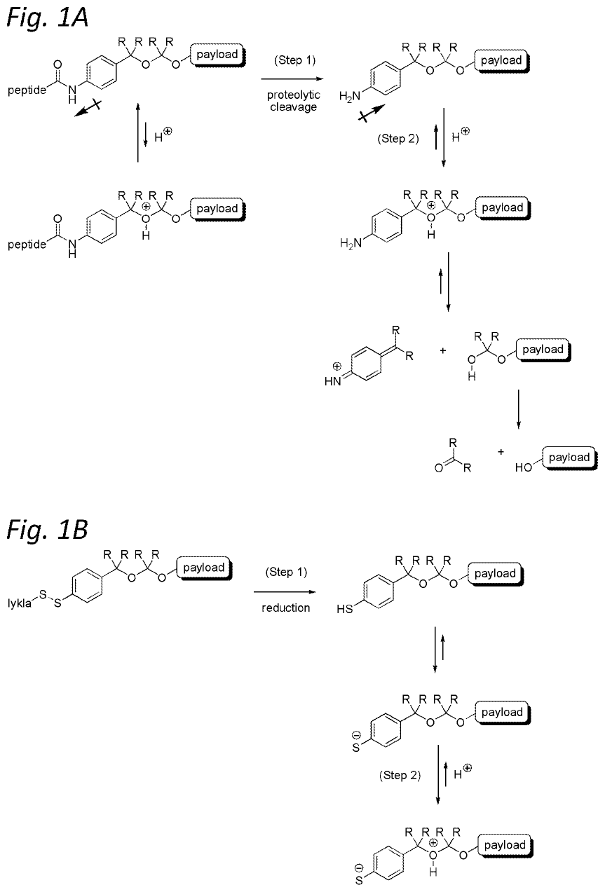

FIG. 2 shows are range of cytotoxic payloads used for ADCs in the field of oncology, with a primary or secondary amino group (highlighted with arrow), which may be acylated with the cleavable linkers of the invention.

FIG. 3 depicts how a payload with a free amino group can be readily converted into a hydroxyacylated derivative thereof by reaction with an activated ester derivative. The active catabolite that will be released after linker cleavage is boxed.

FIG. 4A shows a range of aminobenzoic acid derivatives that may be employed for acylation of an aliphatic amino group of a payload. Substituents X₁ and X₂ are independently selected from the group consisting of F, Cl, Br, I, CF₃ and OMe. FIG. 4B shows other aniline derivatives that may be employed for acylation of an aliphatic amino group of a payload. The phenyl ring in the derivatives of FIG. 4B may be substituted in the same way as those depicted in FIG. 4A, with X₁ and X₂ independently selected from the group consisting of F, Cl, Br, I, CF₃ and OMe.

FIG. 5 provide a range of anilino derivatives of exatecan obtained by acylation of exatecan free amino group.

FIG. 6 illustrates the modification of a payload containing an amino group (exatecan, DX-89510 with a linker possessing a protease-sensitive moiety (Val-Ala-p-aniline), an acid-sensitive moiety (cyclohexyl-substituted O,O-acetal) and a strained alkyne (BCN) for metal-free click chemistry. Incubation of BCN-linker-payload with azido-modified antibody leads to generation of an ADC. Tumour uptake and cellular processing by consecutive hydrolysis by protease and acid will release hydroxyacetyl-exatecan, also known as Dxd.

FIG. 7 shows a representative set of functional groups (F) in a biomolecule, either naturally present or introduced by engineering, which upon reaction with a reactive group lead to connecting group Z. Functional group F may be artificially introduced (engineered) into a biomolecule at any position of choice. The pyridazine Connecting group (bottom line) is the product of the rearrangement of the tetrazabicyclo[2.2.2]octane connecting group, formed upon reaction of tetrazine with alkyne, with loss of N₂.

### DETAILED DESCRIPTION OF THE INVENTION

### Definitions

The verb “to comprise”, and its conjugations, as used in this description and in the claims is used in its non-limiting sense to mean that items following the word are included, but items not specifically mentioned are not excluded. In addition, reference to an element by the indefinite article “a” or “an” does not exclude the possibility that more than one of the element is present, unless the context clearly requires that there is one and only one of the elements. The indefinite article “a” or “an” thus usually means “at least one”.

The compounds disclosed in this description and in the claims may comprise one or more asymmetric centres, and different diastereomers and/or enantiomers may exist of the compounds. The description of any compound in this description and in the claims is meant to include all diastereomers, and mixtures thereof, unless stated otherwise. In addition, the description of any compound in this description and in the claims is meant to include both the individual enantiomers, as well as any mixture, racemic or otherwise, of the enantiomers, unless stated otherwise. When the structure of a compound is depicted as a specific enantiomer, it is to be understood that the invention of the present application is not limited to that specific enantiomer.

The compounds may occur in different tautomeric forms. The compounds according to the invention are meant to include all tautomeric forms, unless stated otherwise. When the structure of a compound is depicted as a specific tautomer, it is to be understood that the invention of the present application is not limited to that specific tautomer.

The compounds disclosed in this description and in the claims may further exist as exo and endo diastereoisomers. Unless stated otherwise, the description of any compound in the description and in the claims is meant to include both the individual exo and the individual endo diastereoisomers of a compound, as well as mixtures thereof. When the structure of a compound is depicted as a specific endo or exo diastereomer, it is to be understood that the invention of the present application is not limited to that specific endo or exo diastereomer.

Furthermore, the compounds disclosed in this description and in the claims may exist as cis and trans isomers. Unless stated otherwise, the description of any compound in the description and in the claims is meant to include both the individual cis and the individual trans isomer of a compound, as well as mixtures thereof. As an example, when the structure of a compound is depicted as a cis isomer, it is to be understood that the corresponding trans isomer or mixtures of the cis and trans isomer are not excluded from the invention of the present application. When the structure of a compound is depicted as a specific cis or trans isomer, it is to be understood that the invention of the present application is not limited to that specific cis or trans isomer.

The compounds according to the invention may exist in salt form, which are also covered by the present invention. The salt is typically a pharmaceutically acceptable salt, containing a pharmaceutically acceptable anion. The term “salt thereof” means a compound formed when an acidic proton, typically a proton of an acid, is replaced by a cation, such as a metal cation or an organic cation and the like. Where applicable, the salt is a pharmaceutically acceptable salt, although this is not required for salts that are not intended for administration to a patient. For example, in a salt of a compound the compound may be protonated by an inorganic or organic acid to form a cation, with the conjugate base of the inorganic or organic acid as the anionic component of the salt.

The term “pharmaceutically accepted” salt means a salt that is acceptable for administration to a patient, such as a mammal (salts with counter ions having acceptable mammalian safety for a given dosage regime). Such salts may be derived from pharmaceutically acceptable inorganic or organic bases and from pharmaceutically acceptable inorganic or organic acids. “Pharmaceutically acceptable salt” refers to pharmaceutically acceptable salts of a compound, which salts are derived from a variety of organic and inorganic counter ions known in the art and include, for example, sodium, potassium, calcium, magnesium, ammonium, tetraalkylammonium, etc., and when the molecule contains a basic functionality, salts of organic or inorganic acids, such as hydrochloride, hydrobromide, formate, tartrate, besylate, mesylate, acetate, maleate, oxalate, etc.

The term “protein” is herein used in its normal scientific meaning. Herein, polypeptides comprising about 10 or more amino acids are considered proteins. A protein may comprise natural, but also unnatural amino acids.

The term “monosaccharide” is herein used in its normal scientific meaning and refers to an oxygen-containing heterocycle resulting from intramolecular hemiacetal formation upon cyclisation of a chain of 5-9 (hydroxylated) carbon atoms, most commonly containing five carbon atoms (pentoses), six carbon atoms (hexose) or nine carbon atoms (sialic acid). Typical monosaccharides are ribose (Rib), xylose (Xyl), arabinose (Ara), glucose (Glu), galactose (Gal), mannose (Man), glucuronic acid (GlcA), N-acetylglucosamine (GlcNAc), N-acetylgalactosamine (GalNAc) and N-acetylneuraminic acid (NeuAc).

The term “antibody” is herein used in its normal scientific meaning. An antibody is a protein generated by the immune system that is capable of recognizing and binding to a specific antigen. An antibody is an example of a glycoprotein. The term antibody herein is used in its broadest sense and specifically includes monoclonal antibodies, polyclonal antibodies, dimers, multimers, multispecific antibodies (e.g. bispecific antibodies), antibody fragments, and double and single chain antibodies. The term “antibody” is herein also meant to include human antibodies, humanized antibodies, chimeric antibodies and antibodies specifically binding cancer antigen. The term “antibody” is meant to include whole immunoglobulins, but also antigen-binding fragments of an antibody. Furthermore, the term includes genetically engineered antibodies and derivatives of an antibody. Antibodies, fragments of antibodies and genetically engineered antibodies may be obtained by methods that are known in the art. Typical examples of antibodies include, amongst others, abciximab, rituximab, basiliximab, palivizumab, infliximab, trastuzumab, efalizumab, alemtuzumab, adalimumab, tositumomab-1131, cetuximab, ibrituximab tiuxetan, omalizumab, bevacizumab, natalizumab, ranibizumab, panitumumab, eculizumab, certolizumab pegol, golimumab, canakinumab, catumaxomab, ustekinumab, tocilizumab, ofatumumab, denosumab, belimumab, ipilimumab and brentuximab.

An “antibody fragment” is herein defined as a portion of an intact antibody, comprising the antigen-binding or variable region thereof. Examples of antibody fragments include Fab, Fab′, F(ab′)₂, and Fv fragments, diabodies, minibodies, triabodies, tetrabodies, linear antibodies, single-chain antibody molecules, scFv, scFv-Fc, multispecific antibody fragments formed from antibody fragment(s), a fragment(s) produced by a Fab expression library, or an epitope-binding fragments of any of the above which immunospecifically bind to a target antigen (e.g., a cancer cell antigen, a viral antigen or a microbial antigen).

An “antigen” is herein defined as an entity to which an antibody specifically binds.

The terms “specific binding” and “specifically binds” is herein defined as the highly selective manner in which an antibody or antibody binds with its corresponding epitope of a target antigen and not with the multitude of other antigens. Typically, the antibody or antibody derivative binds with an affinity of at least about 1×10⁻⁷ M, and preferably 10⁻⁸ M to 10⁻⁹ M, 10⁻¹⁰ M, 10⁻¹¹ M, or 10⁻¹² M and binds to the predetermined antigen with an affinity that is at least two-fold greater than its affinity for binding to a non-specific antigen (e.g., BSA, casein) other than the predetermined antigen or a closely-related antigen.

The term “substantial” or “substantially” is herein defined as a majority, i.e. >50% of a population, of a mixture or a sample, preferably more than 50%, 55%, 60%, 65%, 70%, 75%, 80%, 85%, 90%, 91%, 92%, 93%, 94%, 95%, 96%, 97%, 98%, or 99% of a population.

A “linker” is herein defined as a moiety that connects two or more elements of a compound. For example in an antibody-conjugate, an antibody and a payload are covalently connected to each other via a linker. A linker may comprise one or more linkers and spacer-moieties that connect various moieties within the linker.

A “spacer” or spacer-moiety is herein defined as a moiety that spaces (i.e. provides distance between) and covalently links together two (or more) parts of a linker. The linker may be part of e.g. a linker-construct, the linker-conjugate or a bioconjugate, as defined below.

A “self-immolative group” is herein defined as a part of a linker in an antibody-drug conjugate with a function is to conditionally release free drug at the site targeted by the ligand unit. The activatable self-immolative moiety comprises an activatable group (AG) and a self-immolative spacer unit. Upon activation of the activatable group, for example by enzymatic conversion of an amide group to an amino group or by reduction of a disulfide to a free thiol group, a self-immolative reaction sequence is initiated that leads to release of free drug by one or more of various mechanisms, which may involve (temporary) 1,6-elimination of a p-aminobenzyl group to a p-quinone methide, optionally with release of carbon dioxide and/or followed by a second cyclization release mechanism. The self-immolative assembly unit can part of the chemical spacer connecting the antibody and the payload (via the functional group). Alternatively, the self-immolative group is not an inherent part of the chemical spacer, but branches off from the chemical spacer connecting the antibody and the payload.

An “activatable group” is herein defined as a functional group attached to an aromatic group that can undergo a biochemical processing step such as proteolytic hydrolysis of an amide bond or reduction of a disulphide bond, upon which biochemical processing step a self-immolative process of the aromatic group will be initiated. The activatable group may also be referred to as “activating group”.

“Electron-donating group” is herein defined as a functional group or electropositive atom that increases electron density of an atom or functional group moiety to which it is bonded either inductively and/or through resonance, whichever is more dominant and tends to stabilize cations or electron-poor systems. The electron donating effect is typically transmitted through resonance to other atoms attached to the bonded atom that has been made electron rich by the electron donating group (EDG). Exemplary electron donating groups include, but are not limited to amino groups and certain O-linked substituents as described herein such as —OH and ethers. Depending on its substituents, an aryl or heteroaryl moiety may also be an electron donating group.

“Electron-withdrawing group” is herein defined as a functional group or electronegative atom that reduces electron density of an atom or functional group moiety to which it is bonded either inductively and/or through resonance, and tends to destabilize cations or electron-poor systems. The electron-withdrawing effect is typically transmitted through resonance to other atoms attached to the bonded atom that has been made electron-poor by the electron-withdrawing group (EWG). Exemplary electron-withdrawing groups include, but are not limited to amide groups, nitro groups, azido groups, fluorides, chlorides, bromides, iodides. Depending on its substituents, an aryl or heteroaryl moiety may also be an electron-withdrawing group.

A “bioconjugate” is herein defined as a compound wherein a biomolecule is covalently connected to a payload via a linker. A bioconjugate comprises one or more biomolecules and/or one or more target molecules.

A “biomolecule” is herein defined as any molecule that can be isolated from nature or any molecule composed of smaller molecular building blocks that are the constituents of macromolecular structures derived from nature, in particular nucleic acids, proteins, glycans and lipids. Examples of a biomolecule include an enzyme, a (non-catalytic) protein, a polypeptide, a peptide, an amino acid, an oligonucleotide, a monosaccharide, an oligosaccharide, a polysaccharide, a glycan, a lipid and a hormone.

The term “payload” refers to the moiety that is covalently attached to a targeting moiety such as an antibody, but also to the molecule that is released from the conjugate upon cleavage of the linker. Payload thus refers to the monovalent moiety having one open end which is covalently attached to the targeting moiety via a linker, which is in the context of the present invention referred to as D, and also to the molecule that is released therefrom, which is in the context of the present invention referred to as HX-L²-D, wherein XH is a hydroxy group (if X=O) or an amino group (if X=NR³).

### Compounds According to the Invention

In a first aspect, the invention concerns a compound according to structure (1):

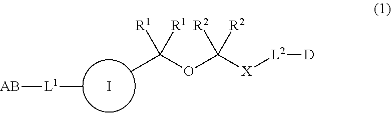

or a salt thereof, wherein
AB is an antibody or a reactive moiety capable of reacting with a functional group on an antibody;
L¹ is a linker;
moiety I is according to formula (2a), (2b), (2c) or (2d):

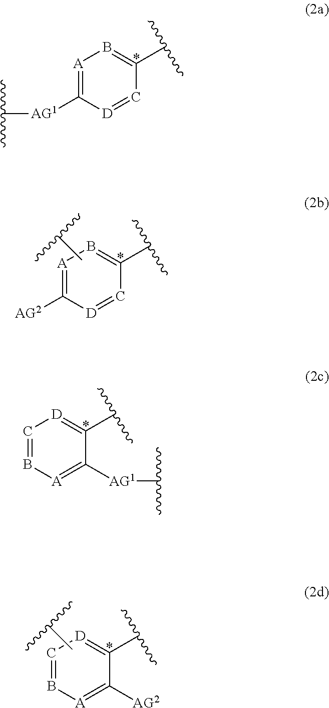

wherein
* indicates the carbon atom connected to C(R¹)₂;
each of A, B, C and D is independently selected from N and CR⁵, wherein R⁵ is H, optionally substituted alkyl, alkoxy, sulfonate, alkylamino or halogen wherein the alkyl may be interrupted by one or more heteroatoms, or in case of formula (2b) and (2d) at least one of A, B, C and D is CR⁵, wherein R⁵ is L¹-AB;
AG¹ is an activating group selected from —S—S— and —NH-peptide-, wherein the peptide contains 1-10 amino acids;
AG² is an activating group selected from the group consisting of —S—S—R¹⁰ and —NH-peptide-R¹¹, —NO₂, —N₃, —OR⁴ and —OC(O)R⁸, wherein the peptide contains 1-10 amino acids, wherein R⁴ is selected from phosphate, sulfate and monosaccharide, R⁸ is a C₂-C₁₀ alkyl group, R¹⁰ is an optionally substituted alkyl or aryl group; and R¹¹ is the optionally protected end group of the peptide (NH₂ or OH, typically NH₂);
each R¹ is independently selected from the group consisting of hydrogen, optionally substituted alkyl, optionally substituted aryl, C(O)OR⁶ and C(O)N(R⁶)₂;
each R² is independently selected from the group consisting of hydrogen, optionally substituted alkyl and optionally substituted aryl, or both occurrences of R² are joined together in a cyclic structure;
X is selected from O and NR³, wherein R³ is selected from the group consisting of H, alkyl, aryl, —C(O)-alkyl, —C(O)-aryl, —C(O)—O-alkyl, —C(O)—O-aryl, —C(O)—N(R⁶)₂, —C(O)—N(R⁶)₂, —S(O₂)—O-alkyl, S(O₂)—O-aryl, —S(O₂)—N(R⁶)₂ and —S(O₂)—N(R⁶)₂;
each R⁶ is independently selected from the group consisting of hydrogen, optionally substituted alkyl and optionally substituted aryl, or both occurrences of R⁶ on the same nitrogen atom are joined into in a cyclic structure;
L² is —C(O)-L³-C(O)— or -L³-C(O)—, wherein L³ is optionally substituted alkylene and/or optionally substituted arylene, wherein the alkylene may optionally be interrupted with 1-3, preferably at most 1, heteroatoms selected from O, NR⁷ and S(O)_{y}, wherein y=0, 1 or 2;
D is a payload.

The invention thus concerns antibody-conjugates, wherein AB is an antibody, as well as precursors to antibody-conjugates, wherein AB is a reactive moiety capable of reacting with a functional group on an antibody. When AB reacts with the functional group on the antibody, a covalent connection between the antibody and payload D is formed, giving the antibody-conjugate. The precursor compound is also referred to as the payload construct. Wherever reference is made to the compound according to the invention, it concerns both the antibody-conjugate and the payload construct.

Reactive moiety AB is capable of reaction with an antibody, which contains an -(L⁴)_{c}-(F)_{d} substituent, wherein L⁴ covalently connects F with the antibody, c is 0 or 1, F is a reactive moiety capable of reacting with reactive moiety AB and d is an integer in the range 1-8. This is further defined in the conjugation process according to the invention.

In a further aspect, the invention concerns a conjugation process for the manufacture of the antibody-conjugate according to the first aspect. The process according to this aspect comprises:
(a) reacting the payload construct according to the first aspect, with an antibody having the structure AB-(L⁴)_{c}-(F)_{d}, wherein
   L⁴ is a linker covalently connecting (L⁴)_{c} and AB;
   c is 0 or 1;
   d is an integer in the range 1-8;
   AB is an antibody;
   F is a functional group capable of reaction with functional group AB,

wherein AB and F form a covalent bond to form a connecting group.

### The Acetal Moiety

The compounds according to the invention are acetals, i.e. they contain an acetal moiety. They may be an O,O-acetal, i.e. wherein X=O, or an O,N-acetal, i.e. wherein X=NR³. In a preferred embodiment, the compound according to the invention is an O,O-acetal and X=O.

R³ is the substituent on the nitrogen atom of the O,N-acetal. R³ is selected from the group consisting of H, alkyl, aryl, —C(O)-alkyl, —C(O)-aryl, —C(O)—O-alkyl, —C(O)—O-aryl, —C(O)—N(R⁶)₂, —C(O)—N(R⁶)₂, S(O₂)—O-alkyl, S(O₂)—O-aryl, —S(O₂)—N(R⁶)₂ and —S(O₂)—N(R⁶)₂. Herein, the alkyl and aryl moieties may optionally be substituted, although in a preferred embodiment they are not substituted. Preferably, R³ is selected from the group consisting of aryl, —C(O)-alkyl, —C(O)-aryl, —C(O)—O-alkyl, —C(O)—O-aryl, —C(O)—N(R⁶)₂, —S(O₂)—O-alkyl, —S(O₂)—O-aryl, —S(O₂)—N(R⁶)₂ and —S(O₂)—N(R⁶)₂, more preferably from the group consisting of —C(O)-alkyl, —C(O)-aryl, —C(O)—O— alkyl and —C(O)—O-aryl.

In a preferred embodiment when X=NR³, there is a carbonyl moiety directly attached to the nitrogen atom, which was found to improve the stability of the compounds of the invention. This carbonyl moiety may be present in R³ or may be present in L². Thus, in one embodiment, R³ is H, alkyl or aryl and L² is —C(O)-L³-C(O)—. In an alternative embodiment, R³ is selected from the group consisting of —C(O)-alkyl, —C(O)-aryl, —C(O)—O-alkyl and —C(O)—O-aryl and L² is -L³-C(O)—.

In the context of R³, it is preferred that the alkyl is C₁-C₁₂ alkyl, more preferably C₁-C₆ alkyl, even more preferably C₁-C₄ alkyl, most preferably C₁-C₂ alkyl.

R⁶ represents the substituents on nitrogen. Each R⁶ is independently selected from the group consisting of hydrogen, alkyl and aryl, wherein alkyl and aryl are optionally substituted. Alternatively, both occurrences of R⁶ on the same nitrogen atom are joined into in a cyclic structure. In the context of R⁶, it is preferred that the alkyl is C₁-C₁₂ alkyl, more preferably C₁-C₆ alkyl, even more preferably C₁-C₄ alkyl, most preferably C₁-C₂ alkyl. In the context of R⁶, the cyclic structure may be a 3-8 membered cycle, preferably a 3-6 membered cycle, more preferably a 5 or 6 membered cycle, most preferably a 6 membered cycle. The cyclic structure may have only carbon ring atoms, in addition to the nitrogen atom of R³, or may contain 1 or 2 additional heteroatoms. Piperidine and morpholine rings are preferred cyclic structures.

R² represents the substituents on the acetal position. These substituents are independently selected from the group consisting of hydrogen, optionally substituted alkyl and optionally substituted aryl. In case R² is alkyl, the alkyl may be unsubstituted or substituted. In case R² is aryl, the aryl may be unsubstituted or substituted. In case both occurrences of R² are joined together in a cyclic structure, this cyclic structure is preferably 3-8 membered cycle, more preferably 3-6 membered, even more preferably 5 or 6 membered, most preferably the cyclic structure is 6 membered. All ring atoms of the cyclic structure may be carbon, or may contain one or more, preferably 1-3, most preferably 1, heteroatom(s), preferably selected from O, NR⁹ and SO₂. Herein, R⁹ may be H or C₁-C₄ alkyl, preferably H or Me. Most preferably, the cyclic structure contains one oxygen atom.

In the context of R², it is preferred that the alkyl is C₁-C₁₂ alkyl, more preferably C₁-C₆ alkyl, even more preferably C₁-C₄ alkyl, most preferably C₁-C₂ alkyl. In a preferred embodiment, R² is independently selected from the group consisting of hydrogen, optionally substituted C₁-C₆ alkyl and optionally substituted C₅-C₆ aryl, or both occurrences of R² are joined together in a 3-6 membered cyclic structure, more preferably R² is independently selected from the group consisting of hydrogen, C₁-C₄ alkyl and phenyl, or both occurrences of R² are joined together in a 5 or 6 membered cyclic structure, even more preferably R² is independently selected from the group consisting of hydrogen, methyl and ethyl, or both occurrences of R² are joined together in a 5 or 6 membered cyclic structure. It is especially preferred that at least one of R² is hydrogen, most preferably both occurrences of R² are hydrogen. In an alternative especially preferred embodiment, one occurrence of R² is methyl and the other occurrence of R² is hydrogen. In an alternative especially preferred embodiment, both occurrences of R² are joined together in a 6 membered cyclic structure, preferably cyclohexyl or oxanyl, most preferably 4-oxanyl.

### The Benzyl Moiety

The compounds according to the invention contain a benzyl moiety, or a benzyl-like moiety, formed by C(R¹)₂ and the aromatic ring that is part of moiety I.

In the aromatic ring, each of A, B, C and D is independently selected from N and CR⁵. Preferably, at least 1 of A, B, C and D is CR⁵, more preferably at least 2 of A, B, C and D are CR⁵, even more preferably at least 3 of A, B, C and D are CR⁵, most preferably each of A, B, C and D are CR⁵, and the aromatic ring is a phenyl ring.

Substituent R⁵ is selected from the group consisting of H, alkyl, alkoxy, sulfonate, alkylamino and halogen. In the context of R⁵, it is preferred that any alkyl (including alkyl present in alkoxy and alkylamino) is C₁-C₁₂ alkyl, more preferably C₁-C₆ alkyl, even more preferably C₁-C₄ alkyl, most preferably C₁-C₂ alkyl. Further, each alkyl moiety may be interrupted by one or more heteroatoms, such as by one heteroatoms for each two carbon atoms. The heteroatoms may be selected from O and NR⁹. Herein, R⁹ may be H or C₁-C₄ alkyl, preferably H or Me. Most preferably, any heteroatom that interrupts the alkyl moiety is O. In case moiety I is represented by formula (2b) or (2d), at least one of A, B, C and D is CR⁵, wherein R⁵ is 1-AB, which is defined hereinbelow.

The aromatic ring is ortho or para substituted with an activating group, which is AG¹ in case the activating group is present in the linkage towards AB, or AG² in case the linkage towards AB is connected to another atom of the aromatic ring. Substitution at the para-position, as in formula (2a) and (2b), is preferred.

R¹ represents the substituents on the benzyl position. These substituents are independently selected from the group consisting of hydrogen, optionally substituted alkyl, optionally substituted aryl, C(O)OR⁶ and C(O)N(R⁶)₂. In case R¹ is alkyl, the alkyl may be unsubstituted or substituted. In case R¹ is aryl, the aryl may be unsubstituted or substituted.

In the context of R¹, it is preferred that the alkyl is C₁-C₁₂ alkyl, more preferably C₁-C₆ alkyl, even more preferably C₁-C₄ alkyl, most preferably C₁-C₂ alkyl. In a preferred embodiment, R¹ is independently selected from the group consisting of hydrogen, optionally substituted C₁-C₆ alkyl and optionally substituted C₅-C₆ aryl, C(O)OR⁶ and C(O)N(R⁶)₂, more preferably R¹ is independently selected from the group consisting of hydrogen, C₁-C₄ alkyl, phenyl, C(O)OR⁶ and C(O)N(R⁶)₂, even more preferably R¹ is independently selected from the group consisting of hydrogen, methyl and ethyl. It is especially preferred that at least one of R¹ is hydrogen, most preferably both occurrences of R¹ are hydrogen.

Substituent R⁶ is selected from the group consisting of H, alkyl and aryl, wherein alkyl and aryl are optionally substituted. Alternatively, both occurrences of R⁶ on the same nitrogen atom are joined into in a cyclic structure. R⁶ is further defined hereinabove in the context of R³.

### The Activating Group

Crucial to the compounds of the present invention is the activating group. This moiety is enzymatically hydrolysed in step 1 of the release process as described further below. The inventors found that hydrolysis of the activating group enhanced the acid-sensitivity of the O,O-acetal or O,N-acetal, which is herein also referred to as activation. In step 2 of the release process, the activated acetal moiety is spliced by acid-mediated hydrolysis such that the payload in the form of HX-L²-D is released. Without enzymatically hydrolysis of the activating group, the acid-sensitivity of the acetal moiety is not enhanced, and it will not be hydrolysed, not even (or hardly, very slowly) in the slightly acidic environment of tumour cells.

Since the payload is released from the conjugate by hydrolysis of the acetal moiety, it is not necessary that the activating group is within the linkage between the benzyl moiety and AB. Hydrolysis of the activating group does not need to splice AB from the payload, as long as it activates the acetal moiety. In order to activate the acetal group, the activating group should be positioned ortho or para from the C(R¹)₂ moiety to which the acetal is connected. From a mechanistic point of view, both these positions are suitable, but less steric hindrance and associated synthesis difficulties are observed with the para position.

The activating group should be divalent when it is positioned within the linkage between the benzyl moiety and AB. In other words, it should have two open attachment points for the remainder of the compound according to the invention. Such an activating group is herein represented as AG¹. In case the activating group is not positioned within the linkage between the benzyl moiety and AB, it should be monovalent, i.e. having one open attachment point for the remainder of the compound according to the invention. Such an activating group is herein represented as AG². Divalent activating groups which are capped on one end with a capping group R⁴ or R⁸ are also suitable as AG².

AG¹ is selected from disulfides and peptides. More specifically, AG¹ is —S—S— or —NH-peptide-. The peptide contains 1-10 amino acids, preferably 2-5 amino acids, most preferably 2 amino acids. The —S—S— bond is a disulfide bond that is susceptible to reduction, thereby cleaving the S—S bond and generating two separate mercapto compounds (having an —SH group), for example treatment of disulfide bond with β-mercaptoethanol, dithiothreitol (DTT), glutathione or cysteine will lead to reduction. The peptide-NH bond is, depending on the specific amino acid sequence of the peptide, susceptible to hydrolysis by specific protease, for example trypsin will hydrolyse the amide bond if the terminal amino acid connected to the —NH bond is lysine or arginine, cathepsin B will cleave if the final two amino acids are valine-alanine or valine-citrulline.

In a preferred embodiment, AG¹ is —NH-peptide-, wherein the peptide contains 1-10 amino acids, preferably 2-5 amino acids. Preferably, the peptide is a dipeptide or a tripeptide, most preferably a dipeptide. Peptide spacers are known in the art. In a preferred embodiment, AG¹ is represented by general structure (4):

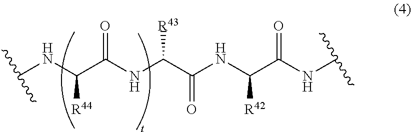

Herein, t is 0, 1, 2 or 3 and R⁴², R⁴³ and each R⁴⁴ are independently selected from the group consisting of amino acid side chains. The wavy lines indicate the connection to the aromatic ring of the benzyl moiety and L¹-AB.

In a preferred embodiment, t=0 or 1, most preferably t=0 and the peptide is a dipeptide. The amino acid side chains are preferably selected from the side chains of alanine, cysteine, aspartic acid, glutamic acid, phenylalanine, glycine, histidine, isoleucine, lysine, leucine, methionine, asparagine, pyrrolysine, proline, gutamine, arginine, serine, threonine, selenocysteine, valine, tryptophan, tyrosine and citrulline. Preferred amino acid side chains are those of alanine, citrulline, lysine, arginine, phenylalanine, isoleucine, leucine, asparagine, tryptophan, valine. Alternatively, R⁴², R⁴³ and R⁴⁴ are preferably independently selected from the group consisting of —(CH₂)₃NHC(NH)NH₂; —CH₂-(1H-imidazol-4-yl); —(CH₂)₄NH₂; —CH₂C(O)OH; —(CH₂)₂C(O)OH; —CH₂OH; —CH(OH)CH₃; —CH₂C(O)NH₂; —(CH₂)₂C(O)NH₂; —CH₂SH; —CH₂SeH; —H; —(CH₂)₃— (joined with the nitrogen atom of same amino acid); —CH₃; —CH(CH₃)CH₂CH₃; —CH₂CH(CH₃)₂; —(CH₂)₂SCH₃; —CH₃Ph; —CH₂-(1H-indol-3-yl); —CH₂(p-Ph)OH; —CH(CH₃)₂; —(CH₂)₄NHC(O)((3-CH₃)-2H-pyrrolin-2-yl); —CH₂CH₂CH₂NHC(O)NH₂. In a preferred embodiment, R⁴², R⁴³ and R⁴⁴ are independently selected from the group consisting of —(CH₂)₃NHC(NH)NH₂; —(CH₂)₄NH₂; —CH₂C(O)NH₂; —CH₃; —CH(CH₃)CH₂CH₃; —CH₂CH(CH₃)₂; —CH₃Ph; —CH₂-(1H-indol-3-yl); —CH(CH₃)₂; —CH₂CH₂CH₂NHC(O)NH₂. In one embodiment, t=0 and R⁴² and R⁴³ are independently selected from the group consisting of —(CH₂)₃NHC(NH)NH₂; —(CH₂)₄NH₂; —CH₂C(O)NH₂; —CH₃; —CH(CH₃)CH₂CH₃; —CH₂CH(CH₃)₂; —CH₂Ph; —CH₂-(1H-indol-3-yl); —CH(CH₃)₂; —CH₂CH₂CH₂NHC(O)NH₂, more preferably from the group consisting of —CH₃; —CH(CH₃)₂; —CH₂CH₂CH₂NHC(O)NH₂. In one embodiment, t=1 and R⁴², R⁴³ and R⁴⁴ are independently selected from the group consisting of —CH₂C(O)NH₂ and —CH₃. In an especially preferred embodiment, AG¹ is represented by general structure (4), wherein t=0, R⁴²=CH₃ or CH₂CH₂CH₂NHC(O)NH₂ and R⁴³=—CH(CH₃)₂.

Alternatively, the peptide is selected from Val-Cit, Val-Ala, Val-Lys, Val-Arg, Phe-Cit, Phe-Ala, Phe-Lys, Phe-Arg, Ala-Lys, Leu-Cit, Ile-Cit, Trp-Cit, Ala-Ala-Asn, Ala-Asn, more preferably Val-Cit, Val-Ala, Val-Lys, Phe-Cit, Phe-Ala, Phe-Lys, Ala-Ala-Asn, even more preferably Val-Cit, Val-Ala, Ala-Ala-Asn. In one embodiment, the peptide is Val-Cit. In one embodiment, the peptide is Val-Ala.

AG² is selected from —S—S—R¹⁰ and —NH-peptide-R¹¹, —NO₂, —N₃, —OR⁴ and —OC(O)R⁸. A nitro group or an azido group may be converted into an amino group upon reduction by a suitable agent such as glutathione. The O-sulfate (R⁴=sulfate) and O-phosphate (R⁴=phosphate) group may be converted into an alcohol with removal of sulfate or phosphate group upon the action of sulfatase or phosphatase enzyme, respectively. Similarly, a monosaccharide-substituted alcohol (R⁴=monosaccharide) may be converted into a free alcohol upon the action of a specific glycosidase, in case monosaccharide and glycosidase are correctly matched, for example a β-glucuronic acid group will be cleaved by β-glucuronidase, a galactose group will be cleaved by a galactosidase, a mannose group will be cleaved by a mannosidase. Finally, ester groups such as —OC(O)R⁸ will be hydrolysed upon the action of esterases. Herein, the peptide is defined as for AG¹, including preferred embodiments thereof. Thus, the —NH-peptide- part of —NH-peptide is preferably according to formula (4) as defined above.

In case AG² is —OR⁴, R⁴ is selected from phosphate, sulfate and monosaccharide. It is preferred that R⁴=monosaccharide, most preferably β-glucuronic acid or galactose. In case AG² is —OC(O)R⁸, R⁸ is a C₂-C₁₂ alkyl group, preferably a C₂-C₆ alkyl group. It is preferred that R⁸ is a short alkyl chain, most preferably a butyl group. In the context of AG², R¹⁰ and R¹¹ are capping groups for respectively the disulphide and the peptide activating groups. R¹⁰ is an optionally substituted alkyl or aryl group, preferably wherein the alkyl is a C₁-C₁₂ alkyl group, preferably a C₁-C₆ alkyl group and preferably wherein the aryl group is phenyl. R¹¹ is an optionally protected end-group of the peptide, typically optionally protected NH₂. Any amine or peptide protection group known in the art can be used, including acetyl (Ac) and carboxybenzyl (Cbz).

### The Linker L¹

Linker L¹ links moiety I with the antibody (after conjugation) or with the reactive group capable of reacting with the antibody (prior to conjugation). The nature of linker L¹ can be very different, depending on the conjugation technique used. The exact structure of linker L¹ is irrelevant for the present invention, as the release process involves the activating group, the benzyl moiety and the acetal moiety, but linker L¹ has no role in that process. The present invention is thus compatible with any type of conjugation, such as activated ester-lysine conjugation, maleimide-cysteine conjugation, alkyne-azide conjugation (copper-catalysed or strain-promoted), reductive alkylation on lysine, oxime ligation, Pictet-Spengler and any other method known to one skilled in the art as bioconjugation, as summarized in G. T. Hermanson, “Bioconjugate Techniques”, Elsevier, 3^{rd} Ed. 2013, incorporated by reference.

L¹ may for example be selected from the group consisting of linear or branched C₁-C₂₀₀ alkylene groups, C₂-C₂₀₀ alkenylene groups, C₂-C₂₀₀ alkynylene groups, C₃-C₂₀₀ cycloalkylene groups, C₅-C₂₀₀ cycloalkenylene groups, C₈-C₂₀₀ cycloalkynylene groups, C₇-C₂₀₀ alkylarylene groups, C₇-C₂₀₀ arylalkylene groups, C₈-C₂₀₀ arylalkenylene groups, C₉-C₂₀₀ arylalkynylene groups. Optionally the alkylene groups, alkenylene groups, alkynylene groups, cycloalkylene groups, cycloalkenylene groups, cycloalkynylene groups, alkylarylene groups, arylalkylene groups, arylalkenylene groups and arylalkynylene groups may be substituted, and optionally said groups may be interrupted by one or more heteroatoms, preferably 1 to 100 heteroatoms, said heteroatoms preferably being selected from the group consisting of O, S(O)_{y} and NR³⁶, wherein y is 0, 1 or 2, preferably y=2, and R³⁶ is independently selected from the group consisting of hydrogen, halogen, C₁-C₂₄ alkyl groups, C₆-C₂₄ (hetero)aryl groups, C₇-C₂₄ alkyl(hetero)aryl groups and C₇-C₂₄ (hetero)arylalkyl groups.

L¹ may contain (poly)ethylene glycoldiamines (e.g. 1,8-diamino-3,6-dioxaoctane or equivalents comprising longer ethylene glycol chains), polyethylene glycol or polyethylene oxide chains, polypropylene glycol or polypropylene oxide chains and 1,z-diaminoalkanes wherein z is the number of carbon atoms in the alkane.

In a preferred embodiment, Linker L¹ comprises a sulfamide group, preferably a sulfamide group according to structure (6):

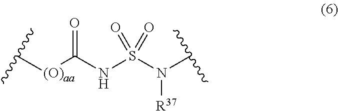

The wavy lines represent the connection to the remainder of the compound, typically to the remainder of L¹. In structure (6), aa=0 or 1, preferably aa=1, and R³⁷ is selected from the group consisting of hydrogen, C₁-C₂₄ alkyl groups, C₃-C₂₄ cycloalkyl groups, C₂-C₂₄ (hetero)aryl groups, C₃-C₂₄ alkyl(hetero)aryl groups and C₃-C₂₄ (hetero)arylalkyl groups, the C₁-C₂₄ alkyl groups, C₃-C₂₄ cycloalkyl groups, C₂-C₂₄ (hetero)aryl groups, C₃-C₂₄ alkyl(hetero)aryl groups and C₃-C₂₄ (hetero)arylalkyl groups optionally substituted and optionally interrupted by one or more heteroatoms selected from O, S and NR³⁸ wherein R³⁸ is independently selected from the group consisting of hydrogen and C₁-C₄ alkyl groups, or R³⁷ is another payload, which is optionally connected to N via a linker, preferably a linker according to the present invention.

In a preferred embodiment, R³⁷ is hydrogen or a C₁-C₂₀ alkyl group, more preferably R³⁷ is hydrogen or a C₁-C₁₆ alkyl group, even more preferably R³⁷ is hydrogen or a C₁-C₁₀ alkyl group, wherein the alkyl group is optionally substituted and optionally interrupted by one or more heteroatoms selected from O, S and NR³⁸, preferably O, wherein R³⁸ is independently selected from the group consisting of hydrogen and C₁-C₄ alkyl groups. In a preferred embodiment, R³⁷ is hydrogen. In another preferred embodiment, R³⁷ is a C₁-C₂₀ alkyl group, more preferably a C₁-C₁₆ alkyl group, even more preferably a C₁-C₁₀ alkyl group, wherein the alkyl group is optionally interrupted by one or more O-atoms, and wherein the alkyl group is optionally substituted with an —OH group, preferably a terminal —OH group. In this embodiment it is further preferred that R³⁷ is a (poly)ethylene glycol chain comprising a terminal —OH group. In another preferred embodiment, R³⁷ is selected from the group consisting of hydrogen, methyl, ethyl, n-propyl, i-propyl, n-butyl, s-butyl and t-butyl, more preferably from the group consisting of hydrogen, methyl, ethyl, n-propyl and i-propyl, and even more preferably from the group consisting of hydrogen, methyl and ethyl. Yet even more preferably, R³⁷ is hydrogen or methyl, and most preferably R³⁷ is hydrogen.

In a preferred embodiment, L¹ is according to structure (7):

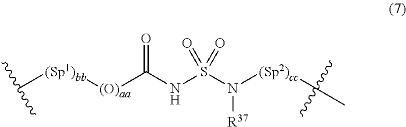

Herein, aa and R³⁷ are as defined above, Sp¹ and Sp² are independently spacer moieties and bb and cc are independently 0 or 1. Preferably, bb=0 or 1 and cc=1, more preferably bb=1 and cc=1. In one embodiment, spacers Sp¹ and Sp² are independently selected from the group consisting of linear or branched C₁-C₂₀₀ alkylene groups, C₂-C₂₀₀ alkenylene groups, C₂-C₂₀₀ alkynylene groups, C₃-C₂₀₀ cycloalkylene groups, C₅-C₂₀₀ cycloalkenylene groups, C₈-C₂₀₀ cycloalkynylene groups, C₇-C₂₀₀ alkylarylene groups, C₇-C₂₀₀ arylalkylene groups, C₈-C₂₀₀ arylalkenylene groups and C₉-C₂₀₀ arylalkynylene groups, the alkylene groups, alkenylene groups, alkynylene groups, cycloalkylene groups, cycloalkenylene groups, cycloalkynylene groups, alkylarylene groups, arylalkylene groups, arylalkenylene groups and arylalkynylene groups being optionally substituted and optionally interrupted by one or more heteroatoms selected from the group of O, S and NR³⁹, wherein R³⁹ is independently selected from the group consisting of hydrogen, C₁-C₂₄ alkyl groups, C₂-C₂₄ alkenyl groups, C₂-C₂₄ alkynyl groups and C₃-C₂₄ cycloalkyl groups, the alkyl groups, alkenyl groups, alkynyl groups and cycloalkyl groups being optionally substituted. When the alkylene groups, alkenylene groups, alkynylene groups, cycloalkylene groups, cycloalkenylene groups, cycloalkynylene groups, alkylarylene groups, arylalkylene groups, arylalkenylene groups and arylalkynylene groups are interrupted by one or more heteroatoms as defined above, it is preferred that said groups are interrupted by one or more O-atoms, and/or by one or more S—S groups.

Most preferably, spacer moieties Sp¹ and Sp², if present, are independently selected from the group consisting of linear or branched C₁-C₂₀ alkylene groups, the alkylene groups being optionally substituted and optionally interrupted by one or more heteroatoms selected from the group of O, S and NR³⁹, wherein R³⁹ is independently selected from the group consisting of hydrogen, C₁-C₂₄ alkyl groups, C₂-C₂₄ alkenyl groups, C₂-C₂₄ alkynyl groups and C₃-C₂₄ cycloalkyl groups, the alkyl groups, alkenyl groups, alkynyl groups and cycloalkyl groups being optionally substituted. In this embodiment, it is further preferred that the alkylene groups are unsubstituted and optionally interrupted by one or more heteroatoms selected from the group of O, S and NR³⁹, preferably O and/or or S—S. Preferred spacer moieties Sp¹ and Sp² thus include —(CH₂)ᵣ—, —(CH₂CH₂)ᵣ—, —(CH₂CH₂O)ᵣ—, —(OCH₂CH₂)ᵣ—, —(CH₂CH₂O)ᵣCH₂CH₂—, —CH₂CH₂(OCH₂CH₂)ᵣ—, —(CH₂CH₂CH₂O)ᵣ—, —(OCH₂CH₂CH₂)ᵣ—, —(CH₂CH₂CH₂O)ᵣCH₂CH₂CH₂— and —CH₂CH₂CH₂—(OCH₂CH₂CH₂)ᵣ, wherein r is an integer in the range of 1 to 50, preferably in the range of 1 to 40, more preferably in the range of 1 to 30, even more preferably in the range of 1 to 20 and yet even more preferably in the range of 1 to 15. More preferably n is 1, 2, 3, 4, 5, 6, 7, 8, 9 or 10, more preferably 1, 2, 3, 4, 5, 6, 7 or 8, even more preferably 1, 2, 3, 4, 5 or 6, yet even more preferably 1, 2, 3 or 4.

In one embodiment, linker L¹ comprises a branching nitrogen atom, which is located in the backbone between AB and moiety I, and which contains a further payload as substituent, which is preferably linked to the branching nitrogen atom via a linker. An example of a branching nitrogen atom is the nitrogen atom NR³⁷ in structure (6), wherein R³⁷ may be connected to a second payload. Alternatively, a branching nitrogen atoms may be located within L¹.

### Reactive Moiety AB

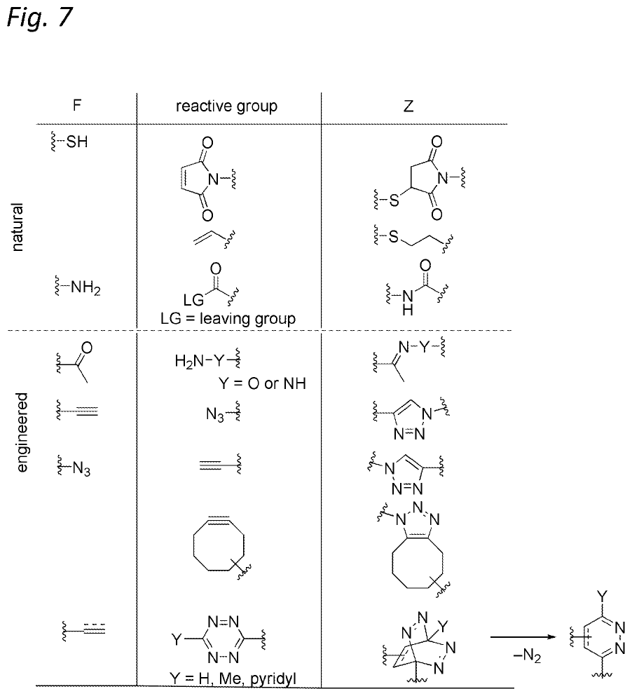

In one embodiment, AB is a reactive moiety capable of reacting with a functional group on an antibody. The payload construct according to the invention comprises such a reactive moiety. Just as for linker L¹, also the specific nature of the reactive moiety is irrelevant for the present invention, as long as it ensures that the payload construct can be conjugated to the antibody. Some exemplary reactive groups are depicted in FIG. 7.

Thus, in order to be reactive in the process according to the second aspect of the invention, the reactive moiety should be capable of reacting with a functional group F present on a biomolecule. In other words, the reactive moiety is complementary to functional group F present in an antibody. Herein, a reactive group is denoted as “complementary” to a functional group when said reactive group reacts with said functional group selectively, optionally in the presence of other functional groups. Complementary reactive and functional groups are known to a person skilled in the art, and are described in more detail below. As such, the compound according to the invention is conveniently used in a conjugation reaction, wherein a chemical reaction between the reactive group and F takes place, thereby forming a bioconjugate comprising a covalent connection between the payload and the antibody.

In a preferred embodiment, the reactive moiety is selected from the group consisting of, optionally substituted, N-maleimidyl groups, ester groups, carbonate groups, protected thiol groups, alkenyl groups, alkynyl groups, tetrazinyl groups, azido groups, phosphine groups, nitrile oxide groups, nitrone groups, nitrile imine groups, diazo groups, ketone groups, (O-alkyl)hydroxylamino groups, hydrazine groups, allenamide groups, triazine groups. In an especially preferred embodiment, the reactive moiety is an N-maleimidyl group, an azide group or an alkynyl group, most preferably the reactive moiety is an alkynyl group. In case the reactive moiety is an alkynyl group, it is preferred that the reactive moiety is selected from terminal alkyne groups, (hetero)cycloalkynyl groups and bicyclo[6.1.0]non-4-yn-9-yl] groups.

### Connecting Group

For the antibody-conjugate according to the present invention, wherein AB is an antibody, linker L¹ links moiety I to the antibody, in which case linker L¹ comprises a connecting group Z, that covalently connects both parts of the conjugate according to the invention. The term “connecting group” herein refers to the structural element, resulting from the reaction between a reactive group and F, connecting one part of a compound and another part of the same compound. As will be understood by the person skilled in the art, the nature of a connecting group depends on the type of conjugation reaction with which the connection between the parts of said compound was obtained. As an example, when the carboxyl group of R—C(O)—OH is reacted with the amino group of H₂N—R′ to form R—C(O)—N(H)—R′, R is connected to R′ via connecting group Z, and Z may be represented by the group —C(O)—N(H)—. Since connecting group Z originates from the reaction between the reactive group and F, it can take any form. Moreover, for the working of the present invention, the nature of connecting group Z is not crucial at all. Whatever form connecting group Z takes, the payload will be liberated at the target site. Some exemplary connecting groups are depicted in FIG. 7.

In one embodiment of the antibody-conjugate, linker L¹ takes the form of (L⁴)ₑ-Z-(L⁵), wherein e is 0 or 1, preferably e=1. Linker L⁴ links the connecting group to antibody AB and originates from the antibody that is functionalized with (L⁴)ₑ-F, and linker L⁵ links the connecting group to moiety I, and originates from the payload construct according to the invention. In a preferred embodiment, the sulfamide group according to formula (6) is contained in L⁵.

Since more than one functional moiety F can be present or introduced in a biomolecule, the conjugate according to the present invention may contain per biomolecule more than one payload, typically the payload to antibody ratio (known in the art as DAR) is in the range 1-10, preferably DAR=1, 2, 3 or 4, more preferably DAR=2 or 4, most preferably DAR=2. For example, in case BM is an antibody, DAR is typically an even integer, in view of the symmetric nature of antibodies. In other words, when one side of the antibody is functionalized with F, the symmetrical counterpart will also be functionalized. Alternatively, in case naturally occurring thiol groups of the cysteine residues of a protein are used as F, the DAR can be anything and may vary between individual conjugates. In such instances of variations, DAR refers to the average drug-to-antibody ratio (payload-to-antibody ratio).

### Antibody AB

In the context of the present invention, biomolecule is preferably selected from the group consisting of proteins (including glycoproteins and antibodies), polypeptides, peptides, glycans, lipids, nucleic acids, oligonucleotides, polysaccharides, oligosaccharides, enzymes, hormones, amino acids and monosaccharides. More preferably, biomolecule B is selected from the group consisting of proteins (including glycoproteins and antibodies), polypeptides, peptides, glycans, nucleic acids, oligonucleotides, polysaccharides, oligosaccharides and enzymes. Most preferably, biomolecule is selected from the group consisting of proteins, including glycoproteins and antibodies, polypeptides, peptides and glycans. In an alternative embodiment, BM refers to a targeting moiety or a targeting biomolecule. In one embodiment of the compounds according to the invention, AB refers to a biomolecule, such as a glycoprotein, preferably an antibody.

The antibody is conjugated to the payload construct by reaction of functional moiety F with the reactive group. F may be a functional moiety that is naturally present in the antibody, such as a disulphide or a lysine side chain, or may be specifically introduced in the antibody to enable site-specific conjugation, e.g. via reduced disulphides or modified glycans. The skilled person is aware of conjugation techniques and understands how to modify the antibody—if needed—to enable conjugation to the payload construct.

### The Payload

D represents the payload, which may also be referred to as molecule of interest. Notably, payload D is distinct from the molecule that is released by the release process, which is HX-L²-D. This released compound is also referred to as payload in the art, and in the contact of the present invention, it may be referred to as released payload. Any payload D can be used in the present invention, as long as an amino group is available for covalent attachment to the acetal moiety. Herein, the amino group may be a primary amino group or a secondary amino group. The payload is covalently attached to C(R²)₂ through L² and the amine group of the payload will be part of the compound according to the invention in the amide bond between D and L². The released payload contains a small linker L² that is present between D and X. A known payload may thus be functionalized with a linker L² via the amino group, which is part of the compound that is released. The amino group of D is preferably an aliphatic amino group. Thus, in one embodiment, D contains an aliphatic amino group, typically an aliphatic primary amino group or an aliphatic secondary amino group, that is used for attachment to L².

Payloads are commonly used in the field of antibody-conjugates, and the type of payload is irrelevant for the functioning of the invention. In a preferred embodiment, the payload is selected from the group consisting of an active substance, a reporter molecule, a polymer, a solid surface, a hydrogel, a nanoparticle, a microparticle and a biomolecule. The skilled person is capable of selecting a suitable payload having a suitable amino group.

The term “active substance” herein relates to a pharmacological and/or biological substance, i.e. a substance that is biologically and/or pharmaceutically active, for example a drug, a prodrug, a diagnostic agent, a protein, a peptide, a polypeptide, a peptide tag, an amino acid, a glycan, a lipid, a vitamin, a steroid, a nucleotide, a nucleoside, a polynucleotide, RNA or DNA. Examples of peptide tags include cell-penetrating peptides like human lactoferrin or polyarginine. An example of a glycan is oligomannose. An example of an amino acid is lysine. When the payload is an active substance, the active substance is preferably selected from the group consisting of drugs and prodrugs. More preferably, the active substance is selected from the group consisting of pharmaceutically active compounds, in particular low to medium molecular weight compounds (e.g. about 200 to about 2500 Da, preferably about 300 to about 1750 Da). In a further preferred embodiment, the active substance is selected from the group consisting of cytotoxins, antiviral agents, antibacterials agents, peptides and oligonucleotides. Most preferably, the payload is a cytotoxin.

The term “reporter molecule” herein refers to a molecule whose presence is readily detected, for example a diagnostic agent, a dye, a fluorophore, a radioactive isotope label, a contrast agent, a magnetic resonance imaging agent or a mass label. A wide variety of fluorophores, also referred to as fluorescent probes, is known to a person skilled in the art. Several fluorophores are described in more detail in e.g. G. T. Hermanson, “Bioconjugate Techniques”, Elsevier, 3d Ed. 2013, Chapter 10: “Fluorescent probes”, p. 395-463, incorporated by reference. Examples of a radioactive isotope label include ^{99m}Tc, ¹¹¹In, ^{114m}In, ¹¹⁵In, ¹⁸F, ¹⁴C, ⁶⁴Cu, ¹³¹I, ¹²⁵I, ¹²³I, ²¹²Bi, ⁸⁸Y, ⁹⁰Y, ⁶⁷Cu, ¹⁸⁶Rh, ¹⁸⁸Rh, ⁶⁶Ga, ⁶⁷Ga and ¹⁰B, which is connected via a ligand, typically a chelating moiety, which contain an amino group for attachment to the acetal moiety. Isotopic labelling techniques are known to a person skilled in the art, and are described in more detail in e.g. G. T. Hermanson, “Bioconjugate Techniques”, Elsevier, 3^{rd} Ed. 2013, Chapter 12: “Isotopic labelling techniques”, p. 507-534, incorporated by reference.

Polymers suitable for use as a payload D in the compound according to the invention are known to a person skilled in the art, and several examples are described in more detail in e.g. G. T. Hermanson, “Bioconjugate Techniques”, Elsevier, 3^{rd} Ed. 2013, Chapter 18: “PEGylation and synthetic polymer modification”, p. 787-838, incorporated by reference. Solid surfaces suitable for use as a payload D are known to a person skilled in the art, and include for example functional surfaces (e.g. a surface of a nanomaterial, a carbon nanotube, a fullerene or a virus capsid). Hydrogels are known to the person skilled in the art. Hydrogels are water-swollen networks, formed by cross-links between the polymeric constituents. See for example A. S. Hoffman, Adv. Drug Delivery Rev. 2012, 64, 18, incorporated by reference. Micro- and nanoparticles suitable for use as a payload D are known to a person skilled in the art. A variety of suitable micro- and nanoparticles is described in e.g. G. T. Hermanson, “Bioconjugate Techniques”, Elsevier, 3d Ed. 2013, Chapter 14: “Microparticles and nanoparticles”, p. 549-587, incorporated by reference. Payload D may also be a biomolecule, which is preferably selected from the group consisting of proteins (including glycoproteins and antibodies), polypeptides, peptides, glycans, lipids, nucleic acids, oligonucleotides, polysaccharides, oligosaccharides, enzymes, hormones, amino acids and monosaccharides.

L² takes the form of —C(O)-L³-C(O)— or -L³-C(O)—. Herein, the C(O) moiety depicted on the right of linker L² is attached to D, in particular to the amino group on the payload, thus forming an amide bond. The left moiety of linker L² is attached to X. Herein, it is preferred that linker L² is -L³-C(O)— in case X is O or NR³, wherein R³ is selected from the group consisting of aryl, —C(O)— alkyl, —C(O)-aryl, —C(O)—O-alkyl, —C(O)—O-aryl, —C(O)—N(R⁶)₂, —C(O)—N(R⁶)₂, —S(O₂)—O-alkyl, S(O₂)—O-aryl, —S(O₂)—N(R⁶)₂ and —S(O₂)—N(R⁶)₂. Likewise, it is preferred that linker L² is —C(O)-L³-C(O)— in case X is NR³, wherein R³ is H or alkyl.

Herein, L³ is an alkylene and/or arylene moiety, wherein the alkylene and arylene are optionally substituted and the alkylene may optionally be interrupted with 1-3, preferably at most 1, heteroatoms selected from O, NR⁷ and S(O)_{y}, wherein y=0, 1 or 2. Preferred heteroatoms for interrupting the alkylene are O and NR⁷. When L³ comprises an alkylene and an arylene moiety, this moiety may also be referred to as an aralkylene moiety. R⁷ is selected from H and C₁-C₄ alkyl. In one embodiment, the alkylene and/or arylene moiety is not substituted. When the alkylene and/or arylene moiety is substituted, the substituents are preferably selected from halide, nitro, cyano, N(R¹²)₂, C₁-C₄ alkyl, halogenated C₁-C₄ alkyl and C₁-C₄ alkoxy, most preferably from halide, CF₃ and methoxy. Herein, R¹² is selected from H and C₁-C₆ alkyl, wherein at least one R¹² is not H. In the context of R¹², the C₁-C₆ alkyl may be interrupted by one or two, preferably by at most one, heteroatom, preferably by O. Also, two occurrences of R¹² on the same nitrogen atom may be joined to form a cyclic structure, preferably a piperidine ring or a morpholine ring. In a preferred embodiment, L³ is an alkylene or arylene moiety, wherein the alkylene and arylene are optionally substituted and the alkylene may optionally be interrupted with one heteroatom selected from O, NR⁷ and S(O)_{y}, wherein y=0, 1 or 2. In a further preferred embodiment, L³ is an alkylene or arylene moiety, wherein the alkylene and arylene are optionally substituted.

In the context of L³, the alkylene is preferably a C₁-C₁₂ alkylene, more preferably C₁-C₆ alkylene, even more preferably C₁-C₄ alkylene, even more preferably C₁-C₂ alkylene, most preferably methylene. In the context of L³, the arylene is preferably a bivalent five- or six-membered aryl ring, more preferably selected from a phenyl ring, a pyridine ring, a pyrimidine ring, a pyridazine ring, a pyrazine ring, a triazole ring, a furan ring, a pyrrole ring, a thiophene ring, an imidazole ring, a pyrazole ring, an oxazole ring, a thiazole ring, a 1,2,4-oxadiazole ring and a 1,3,4-oxadiazole ring. In an especially preferred embodiment, the arylene is a phenylene moiety, which preferably is para-substituted. Preferably, L³ is selected from para-phenylene, meta-phenylene, ortho-phenylene, C₁-C₆ alkylene, para-Ph-O—[C₁-C₆ alkylene], para-Ph-CH₂—O—[C₁-C₆ alkylene] and para-Ph-CH₂—NR⁷—[C₁-C₆ alkylene], which may optionally be substituted. Alkylene moieties are most preferred for L³, such as —(CH₂)_{b}—, wherein b is an integer in the range of 1-10, preferably in the range of 1-4, most preferably b is 1. Thus, most preferably L³ is methylene.

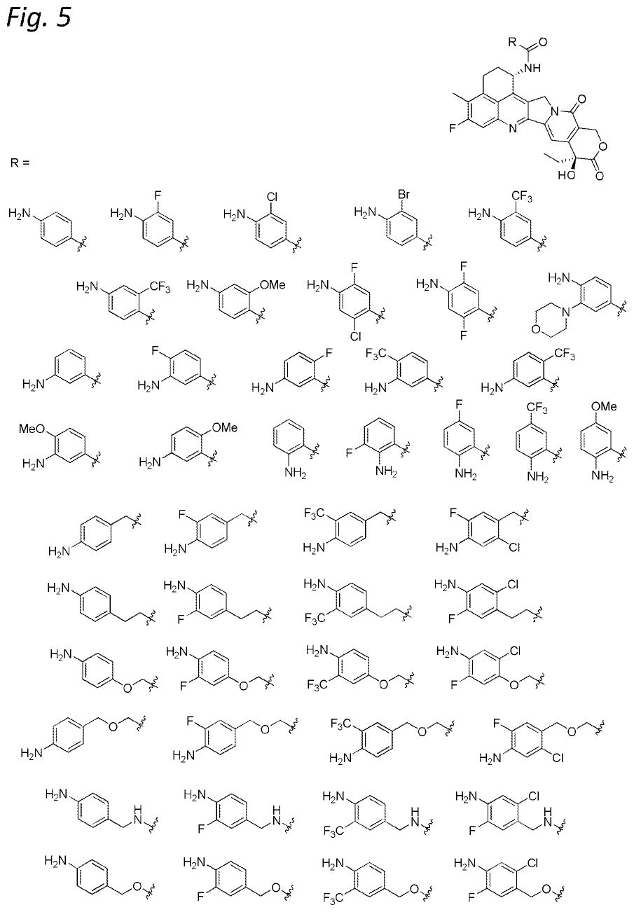

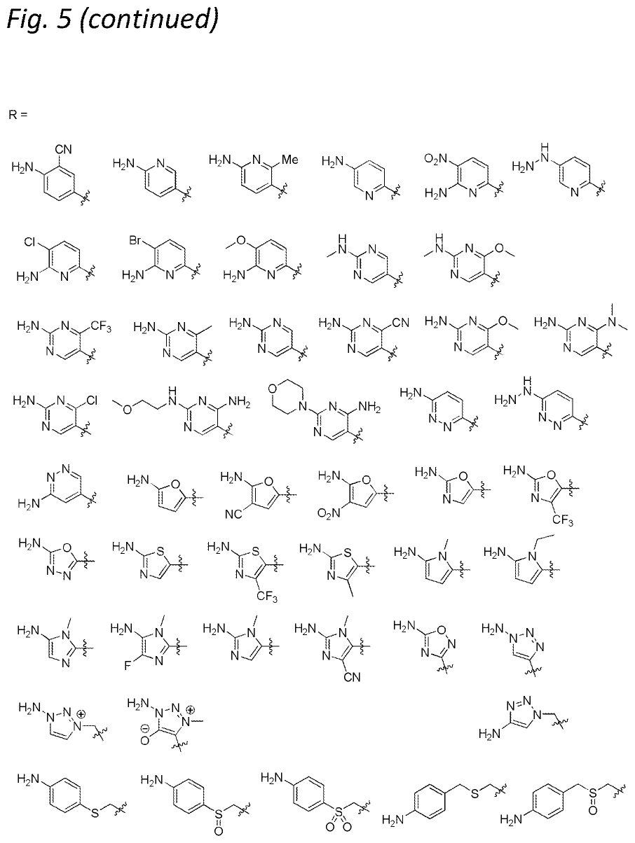

A preferred group of linkers L² is obtained from the benzoic acid derivatives depicted in FIG. 4A or the carboxylic acid derivatives of aniline depicted in FIG. 4B. Herein, the carbonyl moiety of the carboxylic acid group together with the amino group on the payload D form an amide bond, and the primary amino group is connected to the acetal moiety to form an O,N-acetal. Also included in the preferred group of linkers are those obtained from the hydroxyl variants of the compounds depicted in FIGS. 4A and 4B, wherein the NH₂ moiety is replaced by an OH moiety which results in an O,O-acetal. A further group of preferred linkers L³ is depicted in FIG. 5, where they are linked to the payload exatecan. These linkers L³ are equally suitable to be used with other amine-containing payloads. In these structures, the primary amino group is connected to the acetal moiety to form an O,N-acetal. Alternatively, the NH₂ moiety may be replaced by an OH moiety which results in an O,O-acetal.

The nitrogen atom of the amine moiety of the payload D is attached to L², forming an amide bond between the payload and L². The nature of X (O or NR³) can thus be chosen independently from the nature of the attachment point at the payload (amine). As such, amine-containing payloads can be functionalized with an O,O-acetal or with an O,N-acetal. The use of O,O-acetals is preferred, so in a preferred embodiment X=O, more preferably wherein L² is -L³-C(O)—.

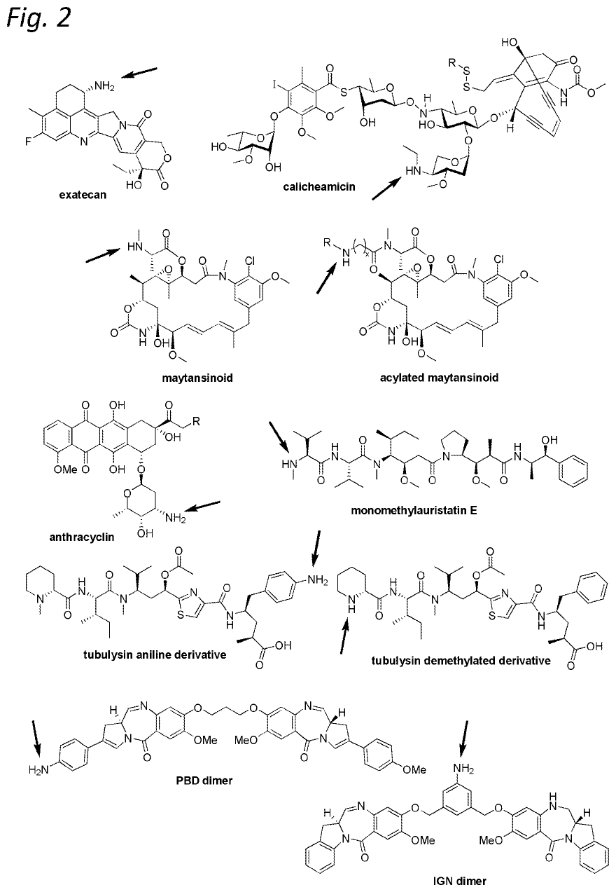

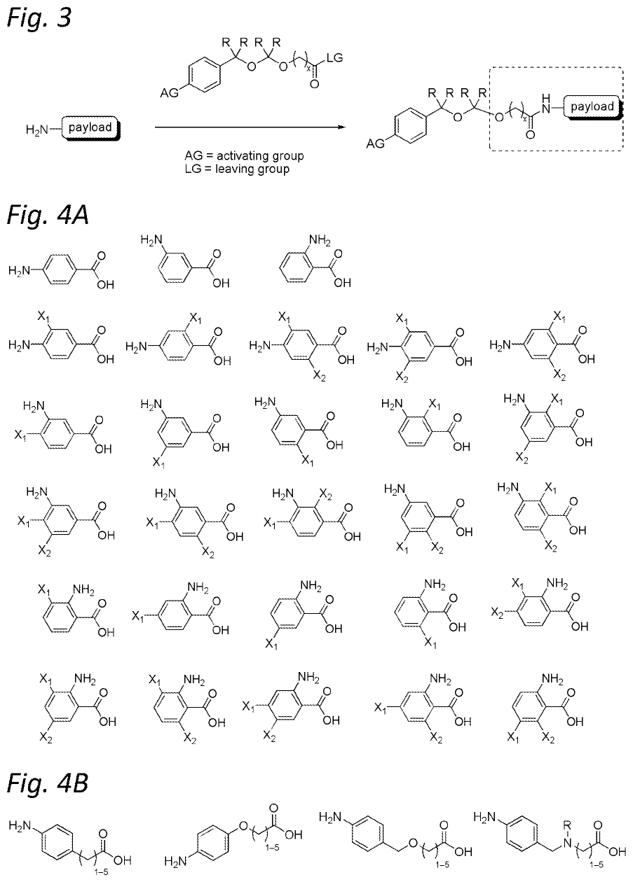

In a particularly preferred embodiment, the payload in unconjugated form contains an amino group (such as present in the payloads illustrated in FIG. 2), which is used for covalent attachment to an acetyl moiety, preferably a hydroxy-acetyl, wherein the hydroxyl group corresponds to X in the compound according to the invention. Amine-containing cytotoxins are known to often suffer from reduced bystander effect, for example exatecan was converted into the hydroxyacetylated derivative DXd to enhance bystander effect. A specific aspect of the present invention concerns an increased bystander effect of amine-containing cytotoxins. In other words, the invention enhances the bystander effect of amine-containing payloads. An enhanced bystander effect is readily achieved by functionalizing the amine moiety of the payload D with linker L² and employing an O,O-acetal (i.e. X=O), as for example illustrated in FIG. 3. As such, the payload that is released (boxed in FIG. 3) has the structure HO-L²-D, wherein the connection between D and L² is formed by an amide bond. Preferably, the released payload has the structure HO—CH₂—(CO)-D. An alternative to hydroxy-acylation of the amino group of the payload comprises first an aminoacylation step with a specific aminobenzoic acid variant (example provided in FIG. 4A) or another carboxylic acid derivative of an aniline (FIG. 4B). A range of such derivatives resulting from acylation of exatecan is exemplified in FIG. 5.

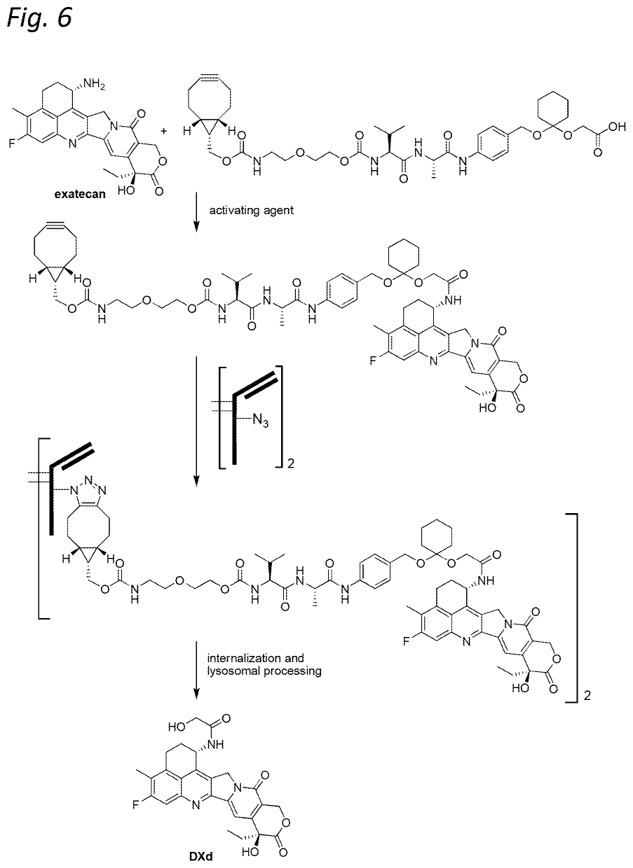

In an especially preferred embodiment, the payload D is a cytotoxin selected from exatecan, calicheamicin or a derivative thereof, a maytansinoid, an anthracyclin, a monomethyl derivative of an auristatin, a tubulysin derivative and C-2 substituted PBD dimer. These cytotoxins contain an amine moiety or an amine moiety is readily introduced. Preferred structures are depicted in FIG. 2, wherein the highlighted nitrogen atom is connected to L² via an amide bond. Preferred structures for D are exatecan, a calicheamicin derivative or a maytansinoid derivative, most preferably an exatecan derivative. In FIG. 6, it is illustrated how exatecan can be converted by a simple acylation step into a linker-drug suitable for bioconjugation to an azido-modified antibody to generate an ADC. Upon treatment of a patient, the ADC will be internalized after recognition of the cancer antigen, leading to consecutive cleavage of peptide and acetal, thereby generating the active catabolite hydroxyacetyl-exatecan (DXd).

### Released Payloads

In one aspect, the invention concerns the payloads that are released by the conjugate according to the invention, or salts thereof. These payloads have the structure HX-L²-D, wherein X and L² are as defined above. In the context of the present aspect, D is according to formula (5a) to (5i). Preferably, X=O. In an especially preferred embodiment, the released payload according to the invention is a calicheamicin according to formula (5a) or a maytansinoid according to formula (5b) or (5c).

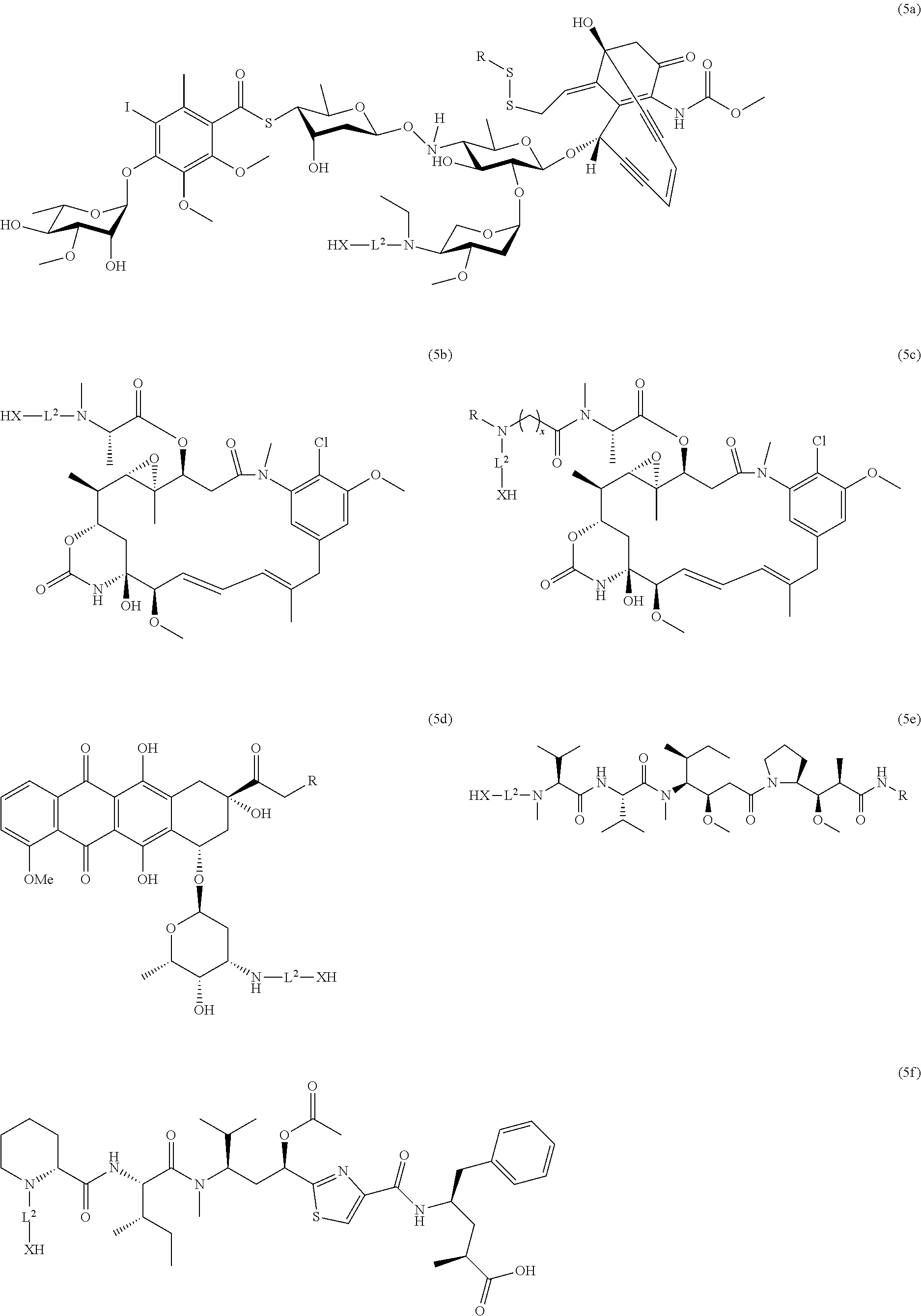

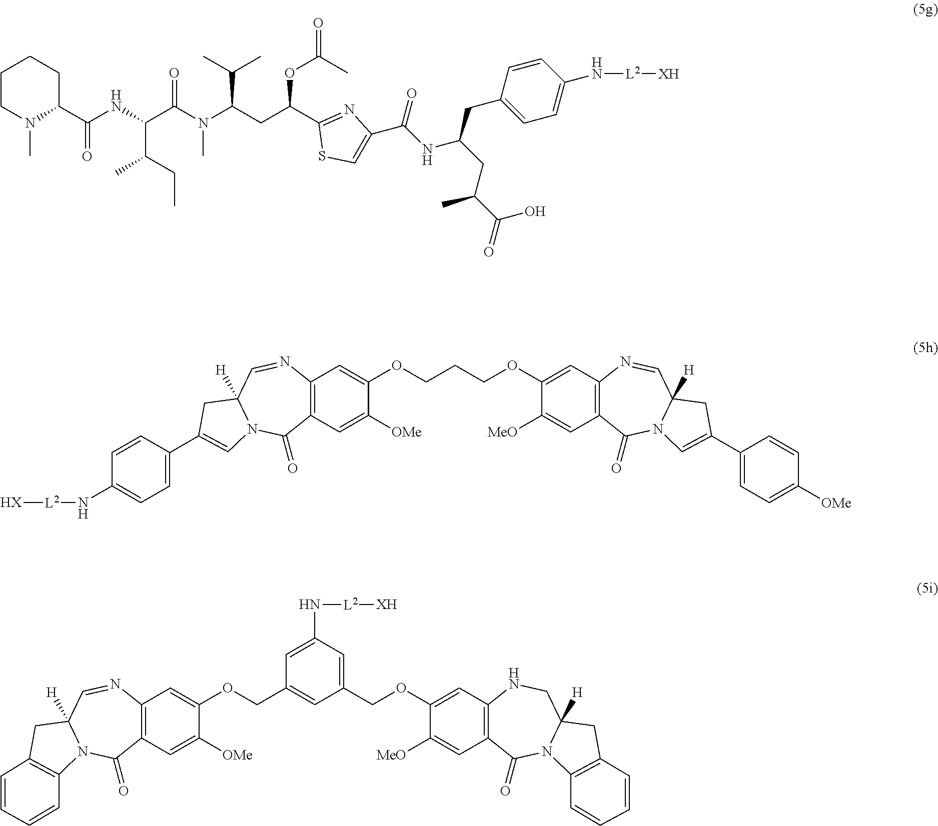

In structures (5a)-(5i), X and L² are as defined above.

In structure (5a), R is —S(C₁-C₁₀ alkyl) or —C(R¹⁶)₂R¹⁷, preferably —SCH₃ or —C(R¹⁶)₂R¹⁷, wherein each R¹⁶ is independently selected from H or optionally substituted C₁-C₆ alkyl and R¹⁷ is selected from H, C₁-C₁₂ alkyl, -L⁶-OR¹⁸, (CH₂)ₛO-L⁶-OR¹⁸ or (CH₂)ₛC(O)NR¹⁹-L⁶-OR¹⁸. Preferably, R¹⁷ is selected from -L⁶-OR¹⁸, (CH₂)ₛO-L⁶-OR¹⁸ or (CH₂)ₛC(O)NR¹⁹-L⁶-OR¹⁸, most preferably R¹⁷=(CH₂)₂O-L⁶-OR¹⁸. Herein, L⁶ is a polar linker having 1-100 optionally substituted backbone atoms selected from C, N, O and S, R¹⁸ is H or methyl, s=1, 2 or 3, preferably s=3. and R¹⁹ is selected from H and -L⁶-OR¹⁸, preferably R¹⁹=H. In one preferred embodiment, R is —SCH₃ or —C(CH₃)₂(R¹⁷). In case R is —S(C₁-C₁₀ alkyl), it is preferred that R is —S(C₁-C₄ alkyl), more preferably wherein the alkyl is selected from methyl, ethyl, propyl, isopropyl and butyl. Most preferably, the alkyl is methyl. L⁶ is a linker having 1-100 backbone atoms, preferably 1-50 backbone atoms, most preferably 3-40 backbone atoms, selected from C, N, O and S. Backbone atoms herein refer to the shortest chain of atoms between the nitrogen atom to which R¹⁷ is connected and the OR¹⁸ moiety. Each of the backbone atoms may be optionally be substituted, preferably with one or two substituents selected from oxo, N(R¹⁸)₂, OR¹⁸, C(O)R¹⁸, C(O)OR¹⁸, C(O)N(R¹⁸)₂. An especially preferred substituent for a carbon or sulphur backbone atom is oxo. Preferably, 0-5, more preferably 0-2, of the backbone atoms is substituted. In one embodiment, 0 of the backbone atoms is substituted. In one embodiment, 2 of the backbone atoms is substituted. It is preferred that L⁶ contains 1-40, preferably 2-30, more preferably 2, 4, 7, 8, 9, 13, 24 or 29 carbon backbone atoms; 0-20, preferably 0-15, more preferably 0, 1, 2, 3, 5, 11 or 13 oxygen backbone atoms, 0-3, preferably 0 or 2, nitrogen backbone atoms; and 0-2, preferably 0 or 1, sulphur backbone atoms.

In structure (5c), R is H or C₁-C₁₂ alkyl, preferably C₁-C₄ alkyl, and x is an integer in the range 1-10.

In structure (5d), R is H or OH.

In structure (5e), R is CH(Me)-CH(OH)-Ph.

The released payloads according to the invention are especially beneficial in the treatment of cancer, since the amino group that is normally present is capped with -L²-OH. The inventors found that such capping of the amino group enhances the bystander effect of the payloads. Furthermore, with the conjugates according to the invention, the inventors have developed a way to specifically release these payloads at the side of a tumour (i.e. in the tumour cell or in the microenvironment of the tumour). As such, the efficacy of both the released payloads of the invention as well as the conjugates according to the invention is increased with respect to prior art payloads and conjugates.

The invention further concerns a method for the treatment of cancer, wherein the released payload according to the invention is released in a tumour cell or in the microenvironment of a tumour cell.

In a preferred embodiment according to the invention, the released payload is according to one of the structures (8a)-(8d):

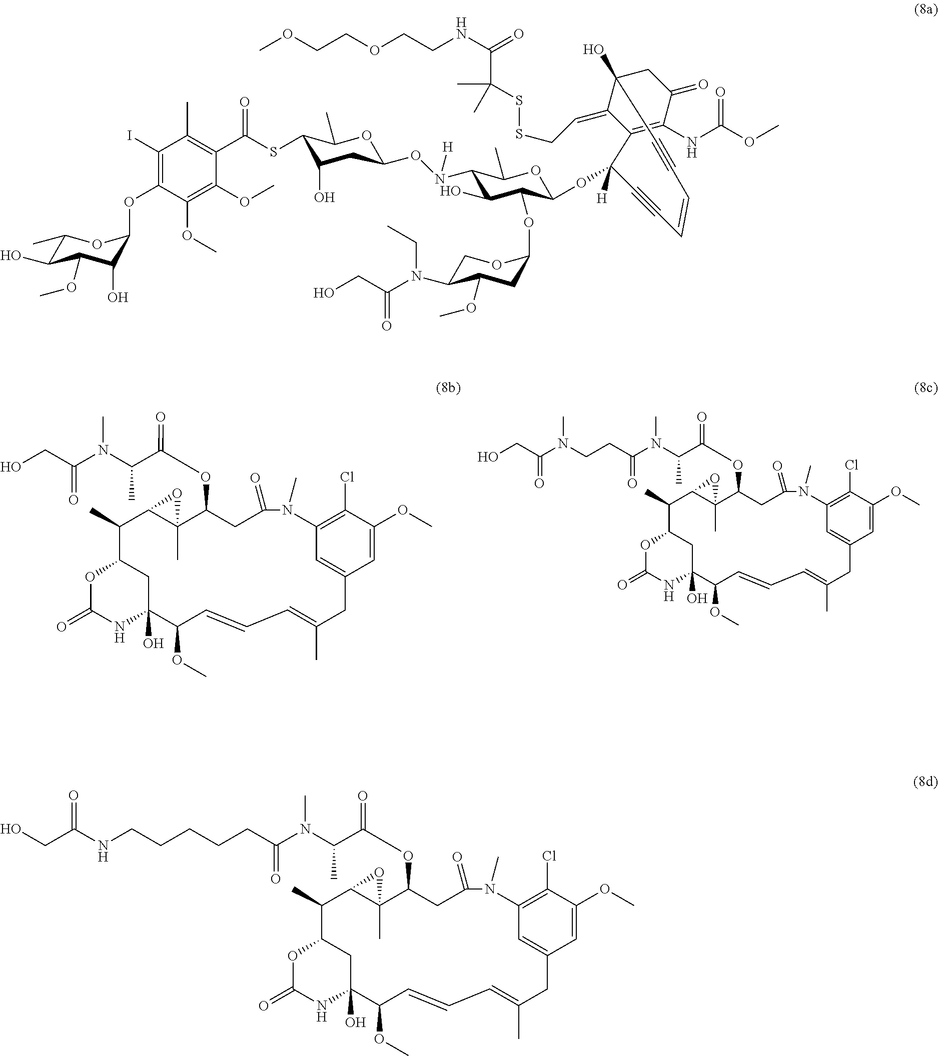

In an alternative preferred embodiment according to the invention, the released payload has the structure HX-L²-D or a salt thereof, wherein X=NH and D is exatecan as depicted in FIG. 5. In other words, the compound according to this preferred embodiment is the exatecan derivative as depicted in FIG. 5, wherein R=L²-NH₂. Herein, L² is as defined above including preferred embodiments thereof. Preferred compounds according to this aspect, i.e. preferred options for R, are depicted in FIG. 5.

### Conjugation Process

The invention further concerns a conjugation process for the manufacture of the conjugate according to the invention, the process comprising the step of reacting the reactive group of the payload construct according to the invention with functional group F of a biomolecule. More specifically, the process according to this aspect comprises:
(a) reacting the payload construct according to structure (1), with an antibody having the structure AB-(L⁴)_{c}-(F)_{d}, wherein
   L⁴ is a linker covalently connecting (L⁴)_{c} and AB;
   c is 0 or 1;
   d is an integer in the range 1-8;
   AB is an antibody;
   F is a functional group capable of reaction with functional group AB,

The present process occurs under condition such that the reactive group of the payload construct is reacted with the functional group F of the biomolecule to covalently link the biomolecule to the payload. In other words, the reactive group reacts with F, forming a covalent connection between the biomolecule and the payload.

The present invention is compatible with any type of conjugation, such as activated ester-lysine conjugation, maleimide-cysteine conjugation, alkyne-azide conjugation (copper-catalysed or strain-promoted), reductive alkylation on lysine, oxime ligation, Pictet-Spengler and any other method known to one skilled in the art as bioconjugation, as summarized in G. T. Hermanson, “Bioconjugate Techniques”, Elsevier, 3d Ed. 2013, incorporated by reference. In one embodiment, the conjugation reaction is selected from the group consisting of activated ester-lysine conjugation, maleimide-cysteine conjugation, alkyne-azide conjugation, reductive alkylation on lysine, oxime ligation and Pictet-Spengler. In a preferred embodiment, the conjugation reaction is via a acylation reaction, a cycloaddition or a Michael reaction. A preferred Michael reaction is the thiol-maleimide addition, most preferably wherein the reactive group is maleimide and F is a thiol group. Preferred cycloadditions are a (4+2)-cycloaddition (e.g. a Diels-Alder reaction) or a (3+2)-cycloaddition (e.g. a 1,3-dipolar cycloaddition). Preferably, the conjugation is the Diels-Alder reaction or the 1,3-dipolar cycloaddition. The preferred Diels-Alder reaction is the inverse-electron demand Diels-Alder cycloaddition. In another preferred embodiment, the 1,3-dipolar cycloaddition is used, more preferably the alkyne-azide cycloaddition, and most preferably wherein the reactive group is an alkyne group and F is an azido group.

### Compositions

The compounds according to the invention can be comprised in a composition, which is also subject of the present invention. As the compounds according to the invention, especially the conjugates according to the invention, are suitably used in medicine, in particular in the treatment of cancer and/or for the targeted delivery of a payload HX-L²-D, the composition is preferably a pharmaceutical composition. Typically, the pharmaceutical composition contains the compounds according to the invention in a pharmaceutically effective amount. The compound comprised in the pharmaceutical composition is preferably the conjugate according to structure (1).

### Applications

The invention further concerns the use of the compound according to the invention for preparing a conjugate according to the invention. More generally, the compounds according to the first aspect can be used for the bioorthogonal labelling or imaging of biomolecules (such as for example proteins, lipids, glycans and the like), proteomics, solid surfaces and other materials. Herein, the biomolecule, proteomic, solid surface or other material contains a reactive moiety F, which may be naturally present or may be engineered according to methods known to the skilled person. By reaction of reactive moiety AB with reactive moiety F, the biomolecule, proteomic, solid surface or other material is covalently labelled with the payload.

The conjugate according to the present invention can be used in medicine. Hence, in a further aspect, the invention also concerns a method of treating a subject in need thereof, comprising administering the conjugate according to the second aspect to the subject. The method according to this aspect can also be worded as the conjugate according to the invention for use in treatment. The method according to this aspect can also be worded as use of the conjugate according to the invention for the manufacture of a medicament. Herein, administration typically occurs with a therapeutically effective amount of the conjugate according to the invention.

The invention further concerns a method for the treatment of a specific disease in a subject in need thereof, comprising the administration of the conjugate according to the invention as defined above. The specific disease may be selected from cancer, a viral infection, a bacterial infection, a neurological disease, an autoimmune disease, an eye disease, hypercholesterolaemia and amyloidosis, more preferable from cancer and a viral infection, most preferably the disease is cancer. The subject in need thereof is typically a cancer patient. The use of conjugates, such as antibody-drug conjugates, is well-known in such treatments, especially in the field of cancer treatment, and the bioconjugates according to the invention are especially suited in this respect. In the method according to this aspect, the conjugate is typically administered in a therapeutically effective amount. The present aspect of the invention can also be worded as a conjugate according to the invention for use in the treatment of a specific disease in a subject in need thereof, preferably for the treatment of cancer. In other words, this aspect concerns the use of a conjugate according to the invention for the preparation of a medicament or pharmaceutical composition for use in the treatment of a specific disease in a subject in need thereof, preferably for use in the treatment of cancer.

The invention further concerns a method for the targeted delivery of a payload HX-L²-D, comprising administering the conjugate according to the invention to a subject, typically for the treatment of a specific disease, preferably cancer, a viral infection, a bacterial infection, a neurological disease, an autoimmune disease, an eye disease, hypercholesterolaemia, or amyloidosis, more preferable the disease is cancer or a viral infection, most preferably the disease is cancer. The present aspect of the invention can also be worded as a conjugate according to the invention for use in the targeted delivery of the payload. In other words, this aspect concerns the use of a conjugate according to the invention for the preparation of a medicament or pharmaceutical composition for use in the targeted delivery of the payload. Targeted delivery may also occur via administration to a sample taken from a subject. In other words, the invention concerns a method of targeting a cell, comprising contacting the cell with a compound according to the invention, wherein AB is an antibody that specifically targets the cell or a receptor expressed on the cell. Herein, the cell or cellular receptor is typically present in a subject or a sample taken from a subject, preferably wherein the subject is a cancer patient.

The invention further concerns a method of enhancing the bystander effect of an amino-containing payload, typically an amino-containing cytotoxin. The enhancement of the bystander effect is achieved by conversion of the amino group into an hydroxyl group, such that the payload that is released has the structure HO-L²-D. The present inventors have developed a conjugate that is able to release such an hydroxyl-containing payload with enhanced bystander effect with respect to the parent amine-containing payload.

The invention generally concerns a method wherein the compound according to the invention is contacted with a cell, wherein AB is an antibody that specifically targets the cell or a receptor expressed on the cell, and wherein a payload having formula HX-L²-D is released near or in the cell. Herein, the cell or cellular receptor is typically present in a subject or a sample taken from a subject, preferably wherein the subject is a cancer patient. Release involves the hydrolysis of activating group AG¹ or AG² by the action of a proteolytic enzyme near or in the cell, which lowers the pH stability of the acetal moiety, and subsequent protonation of the acetal affords release of the payload having formula HX-L²-D. Such the lowering in pH stability of the acetal moiety may be defined as an increase in rate of hydrolysis at pH 7.4 of at least 2-fold, preferably at least 5-fold, most preferably at least 10-fold. During the contacting, the environment near or in the cell is below physiological pH, preferably in the range of 4.5-6.8.

Administration in the context of the present invention refers to systemic administration. Hence, in one embodiment, the methods defined herein are for systemic administration of the conjugate according to the second aspect. In view of the targeting moiety, the conjugates can be systemically administered and yet the payload will only be locally effectuated, in view of the cleavable linker, which will only cleave in or near the tissue of interest (e.g. a tumour). Systemic administration has a great advantage over local administration, as the drug may also reach tumour metastasis not detectable with imaging techniques and it may be applicable to hematological tumours.

### EXAMPLES

### Example 1. Synthesis of Compound 2

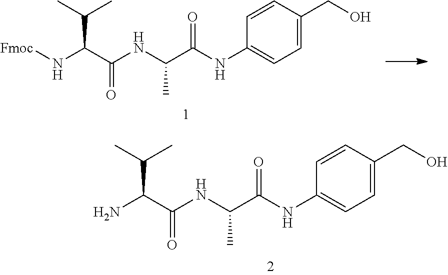

To a colourless solution of 9-fluorenylmethyloxycarbonyl-valinyl-alanyl-4-aminobenzyl-alcohol (1) (502 mg, 973 μmol) in DMF (5.2 mL) was added piperidine (260 μL, 2.63 mmol). The reaction mixture was stirred at room temperature for 75 minutes and then conc. in vacuo. The residue was purified by silica gel chromatography (1%→20% MeOH in DCM) affording the product 2 (88% pure by NMR, 333 mg, quant.) as a colorless oil. ¹H-NMR (400 MHz, CDCl₃) δ (ppm) 7.42 (d, J=8.6 Hz, 2H), 7.19 (d, J=8.6 Hz, 2H), 4.45 (s, 2H), 4.40 (q, J=7.0 Hz, 1H), 3.05 (d, J=5.6 Hz, 1H), 1.94-1.80 (m, 1H), 1.33 (d, J=7.2 Hz, 3H), 0.87 (d, J=6.9 Hz, 3H), 0.81 (d, J=6.8 Hz, 3H).

### Example 2. Synthesis of Compounds 4 and 5

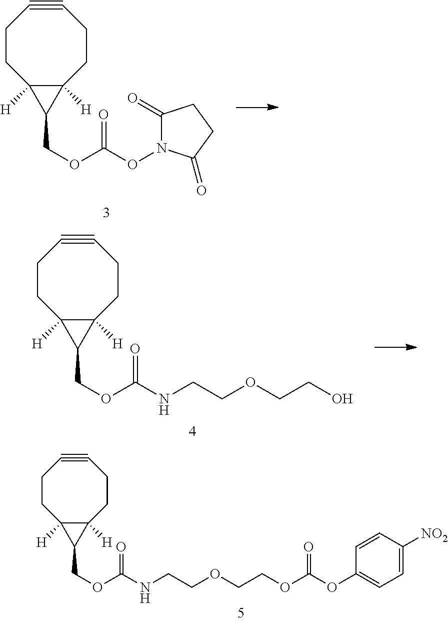 [SMILES: O=C(OC[C@@H]1[C@@H]2CCC#CCC[C@@H]21)ON1C(=O)CCC1=O ; O=C(NCCOCCO)OC[C@@H]1[C@@H]2CCC#CCC[C@@H]21 ; O=C(NCCOCCOC(=O)Oc1ccc([N+](=O)[O-])cc1)OC[C@@H]1[C@@H]2CCC#CCC[C@@H]21]

(1R,8S,9S)-bicyclo[6.1.0]non-4-yn-9-ylmethyl N-succinimidyl carbonate (3) (16.35 g, 56.13 mmol, 1 eq.) was dissolved in DCM (400 ml). 2-(2-aminoethoxy)ethanol (6.76 ml, 67.35 mmol, 1.2 eq.) was then added followed by triethylamine (23.47 ml, 168.39 mmol, 3 eq.). The resulting pale yellow solution was stirred at rt for 90 min. The reaction mixture was concentrated in vacuo and the residue was co-evaporated once with acetonitrile (400 mL). The resulting oil was dissolved in EtOAc (400 mL) and washed three times with water (200 mL). The organic layer was concentrated in vacuo and the residue was purified by flash column chromatography over silicagel (50%→88% EtOAc in heptane) to give 4 as a pale yellow oil (11.2 g, 39.81 mmol, 71%). ¹H-NMR (400 MHz, CDCl₃) δ 5.14-4.89 (bs, 1H). 4.17 (d, J=8.0 Hz, 2H), 3.79-3.68 (m, 2H), 3.64-3.50 (m, 4H), 3.47-3.30 (m, 2H), 2.36-2.14 (m, 6H), 2.03-1.84 (bs, 1H), 1.68-1.49 (m, 2H), 1.37 (quintet, J=8.0 Hz, 1H), 1.01-0.89 (m, 2H).

To a solution of N-(1R,8S,9s)-bicyclo[6.1.0]non-4-yn-9-ylmethyloxycarbonyl 1-amino-3-oxapentan-5-ol (4) (533 mg, 1.92 mmol) in DCM (30 mL) were added 4-nitrophenyl chloroformate (594 mg, 2.95 mmol) and Et₃N (1.13 mL, 824 mg, 5.91 mmol). The resulting mixture was stirred for 2 h and concentrated. The residue was purified by silica gel chromatography (20%→50% EtOAc in heptane). The desired product 5 was obtained as a slightly yellow oil (723 mg, 1.62 mmol, 82%). ¹H NMR (400 MHz, CDCl₃) δ (ppm) 8.32-8.25 (m, 2H), 7.43-7.37 (m, 2H), 5.05 (bs, 1H), 4.46-4.41 (m, 2H), 4.15 (d, J=8.0 Hz, 2H), 3.80-3.75 (m, 2H), 3.60 (t, J=5.1 Hz, 2H), 3.41 (q, J=5.3 Hz, 2H), 2.35-2.15 (m, 6H), 1.65-1.47 (m, 2H), 1.35 (quintet, J=8.3 Hz, 1H), 1.01-0.88 (m, 2H).

### Example 3. Synthesis of Compounds 6 and 7

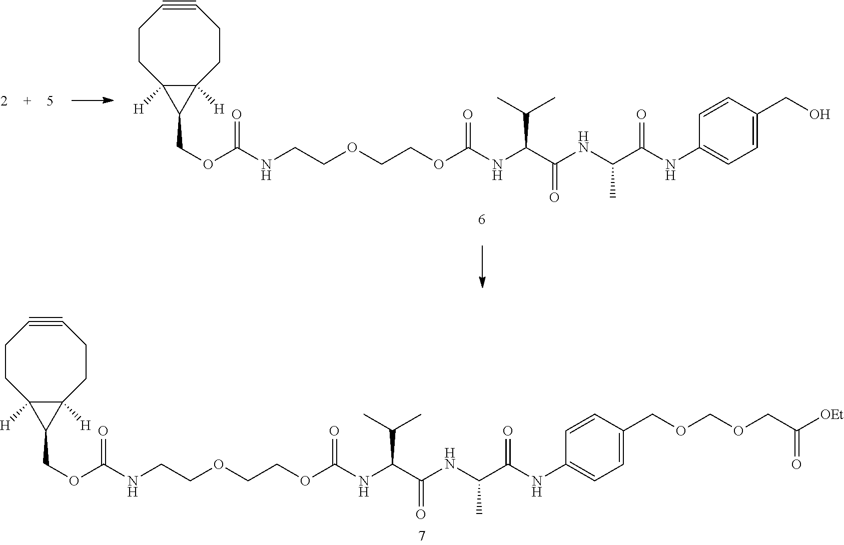 [SMILES: CC(C)[C@H](NC(=O)OCCOCCNC(=O)OC[C@@H]1[C@@H]2CCC#CCC[C@@H]21)C(=O)N[C@@H](C)C(=O)Nc1ccc(CO)cc1 ; CCOC(=O)COCOCc1ccc(NC(=O)[C@H](C)NC(=O)[C@@H](NC(=O)OCCOCCNC(=O)OC[C@@H]2[C@@H]3CCC#CCC[C@@H]32)C(C)C)cc1]

To a solution of 5 (45 mg, 0.10 mmol) in DMF (2 mL) were added a solution of va-PAB-OH (2) (49 mg, 0.13 mmol) in DMF (0.39 mL) and Et₃N (42 μL, 30 mg, 0.30 mmol). The mixture was left for 2.5 h, diluted with DMF (0.5 mL) and concentrated. The residue was purified by silica gel chromatography (0%→15% MeOH in DCM). The desired product was obtained as a white solid (48 mg, 0.080 mmol, 80%). LCMS (ESI+) calculated for C₃₁H₄₅N₄O₈⁺ (M+Na⁺) 601.32. found 601.56.

To a mixture of 6 (34 mg, 57 μmol) and DCM (0.7 mL) were added 2-chloromethoxy)acetic acid ethyl ester (86 mg, 0.57 mmol) and Dipea (99 μL, 73 mg, 0.57 mmol). After addition of additional DCM (5.0 mL) and sonication, the reaction mixture was concentrated at 40° C. and 600 mbar till a volume of 0.43 mL. The mixture was diluted with DCM (0.27 mL). The mixture was left for 20 h and purified by silicagel chromatography (0%→10% MeOH in DCM). The desired product 7 was obtained as a colourless film (27 mg, 38 μmol, 67%). LCMS (ESI+) calculated for C₃₆H₅₃N₄O₁₁+ (M+Na⁺) 717.37. found 717.68.

### Example 4. Synthesis of Compounds 8 and 10

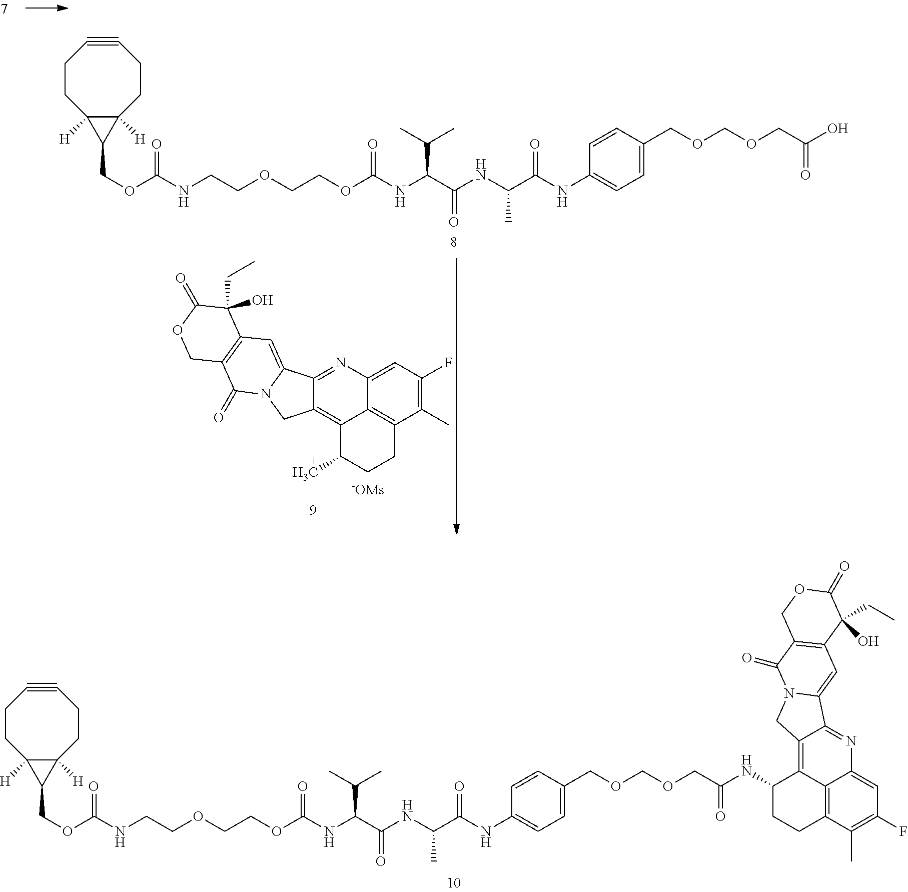 [SMILES: CC(C)[C@H](NC(=O)OCCOCCNC(=O)OC[C@@H]1[C@@H]2CCC#CCC[C@@H]21)C(=O)N[C@@H](C)C(=O)Nc1ccc(COCOCC(=O)O)cc1 ; CC[C@@]1(O)C(=O)OCc2c1cc1n(c2=O)Cc2c-1nc1cc(F)c(C)c3c1c2[C@@H](C)CC3 ; CC[C@@]1(O)C(=O)OCc2c1cc1n(c2=O)Cc2c-1nc1cc(F)c(C)c3c1c2[C@@H](NC(=O)COCOCc1ccc(NC(=O)[C@H](C)NC(=O)[C@@H](NC(=O)OCCOCCNC(=O)OC[C@@H]2[C@@H]4CCC#CCC[C@@H]42)C(C)C)cc1)CC3 ; "-OMs"]

To a solution of 7 (10 mg, 14 μmol) in DMF (7.0 mL) was added a 4 mM aqueous solution of NaOH (3.5 mL). After 75 min, additional 4 mM aqueous NaOH (3.5 mL) was added. After 2.5 h the reaction mixture was poured out in a stirred mixture of potassium phosphate buffer (pH 3.0, 0.5 M, 50 mL) and dichloromethane (20 mL). The layers were separated and the organic phase was dried (Na₂SO₄) and concentrated. The residue was dissolved in DMF (1.0 mL) and used crude in the next step.

To the crude solution of 8 in DMF (0.21 mL) were added a suspension of exatecan mesylate (9) (1.0 mg, 2.2 μmol) in DMF (19 μL), a solution of EDCl (0.42 mg, 2.2 μmol) in a mixture of DMF and DCM (1/1, 20 μL) and Et₃N (0.92 μL). After 18 h, another 0.21 mL of the solution of 8 was then mixed with a solution of EDCl (0.42 mg, 2.2 μmol) in DMF (18 μL) and a solution of N-hydroxysuccinimide (0.24 mg, 2.1 μmol) in DMF (5.6 μL) and added to the reaction mixture. After an additional 24 h, the reaction mixture was purified by RP-HPLC (30%→90% MeCN (1% AcOH) in H₂O (1% AcOH). The desired product 10 was obtained as a white film (1.0 mg, 0.90 μmol, 41%). LCMS (ESI+) calculated for C₅₈H₆₉FN₇O₁₄⁺ (M+H⁺) 1106.49. found 1106.73.

### Example 5. Synthesis of Compound 13 (According to Matysiak et al, Helv. Chim. Acta, 1998, 81, 1545-1566, Incorporated by Reference)

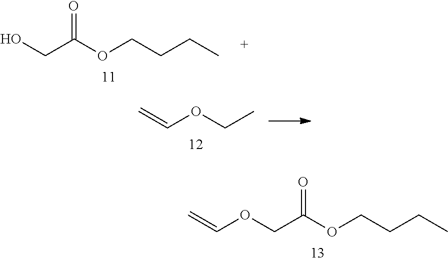

To a cooled (0° C.) solution of butyl glycolate (11) (0.66, 5.0 mmol) in ethyl vinyl ether (12) (1.4 mL, 1.1 g, 15 mmol) was added HgOAc₂ (1.6 g, 5.0 mmol). The mixture was stirred for 21 h and diluted with Et₂O (40 mL) and washed with aqueous saturated NaHCO₃ (2×20 mL). The organic phase was concentrated and the residue was purified by silica gel chromatography (pentane→15% Et₂O in pentane). The desired product 13 was obtained as a colourless liquid (0.17 g, 1.1 mmol, 21%). ¹H NMR (400 MHz, CDCl₃) δ (ppm) 6.50 (dd, J=14.3 Hz, J=6.8 Hz, 1H), 4.32 (s, 2H), 4.21 (dd, J=14.3 Hz, J=2.8 Hz, 1H), 4.20 Hz (t, J=6.7 Hz, 2H), 4.13 (dd, J=6.8 Hz, J=2.8 Hz, 1H), 1.70-1.60 (m, 2H), 1.45-1.33 (m, 2H), 0.94 (t, J=7.4 Hz).

### Example 6. Synthesis of Compound 14

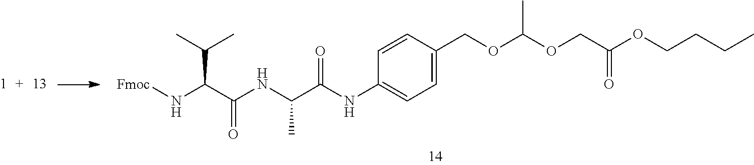

To a solution of Fmoc-va-PAB-OH (1) (20 mg, 0.039 mmol) in anhydrous DMF (1 mL) were added 13 (61 mg, 0.39 mmol) and p-toluenesulfonic acid hydrate (3.0 mg, 0.016 mmol). The mixture was stirred for 3 d and concentrated. The residue was purified by silica gel chromatography (30%→70% EtOAc in heptane). The desired product 14 was obtained as a colorless film (11 mg, 0.016 mmol, 42%). LCMS (ESI+) calculated for C₃₈H₄₇N₃NaO₈⁺ (M+Na⁺) 696.33, found 696.36.

### Example 7. Synthesis of Compounds 15 and 16

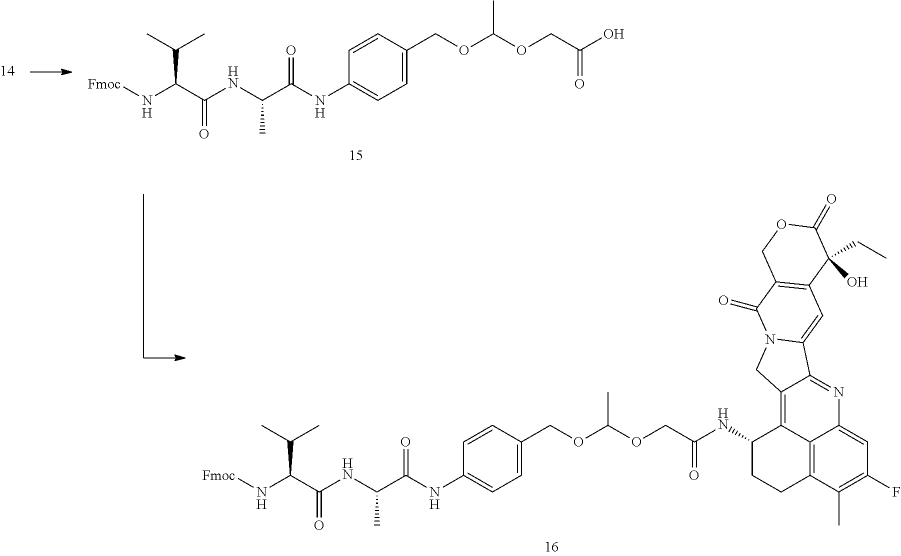

To a solution of 14 (11 mg, 0.016 mmol) in 1,2-dichloroethane (2 mL) was added Me₃SnOH (15 mg, 0.082 mmol) and the mixture was heated to 80° C. After 6 h, 1,2-dichloroethane (2 mL) was added and the mixture was stirred at 80° C. for another 18 h. The mixture was then allowed to come r.t. and stirred for another 24 h. Dichloromethane (2 mL) was added and the mixture was purified by silica gel chromatography (0%→30% MeOH in DCM). The desired product 15 was obtained as a white solid (2.6 mg, 0.0042 mmol, 26%). LCMS (ESI+) calculated for C₃₄H₃₉N₃NaO₈⁺ (M+Na⁺) 640.26. found 640.34.

To a suspension of exatecan mesylate (9) (2.2 mg, 4.2 μmol) in DMF (196 μL) were added a solution of N-hydroxysuccinimide (0.48 mg, 4.2 μmol) in DMF (9.2 μL), a solution of 15 (2.6 mg, 4.2 μmol) in DMF (300 μL), a solution of N,N-diisopropylethylamine (0.54 mg, 0.73 μL, 4.2 μmol) in DMF (7.3 μL) and solution of N,N′-dicyclohexylcarbodiimide (1.1 mg, 5.5 μmol) in DMF (35 μL). The resulting mixture was left for 3 days, diluted with dichloromethane (0.8 mL) and purified by silica gel chromatography (0%→10% MeOH in DCM). The desired product 16 was obtained as a colourless film (2.2 mg, 2.1 μmol, 50%). LCMS (ESI+) calculated for C₅₈H₆₀FN₆O₁₁⁺ (M+H⁺) 1035.43. found 1035.46.

### Example 8. Synthesis of Compounds 17 and 18

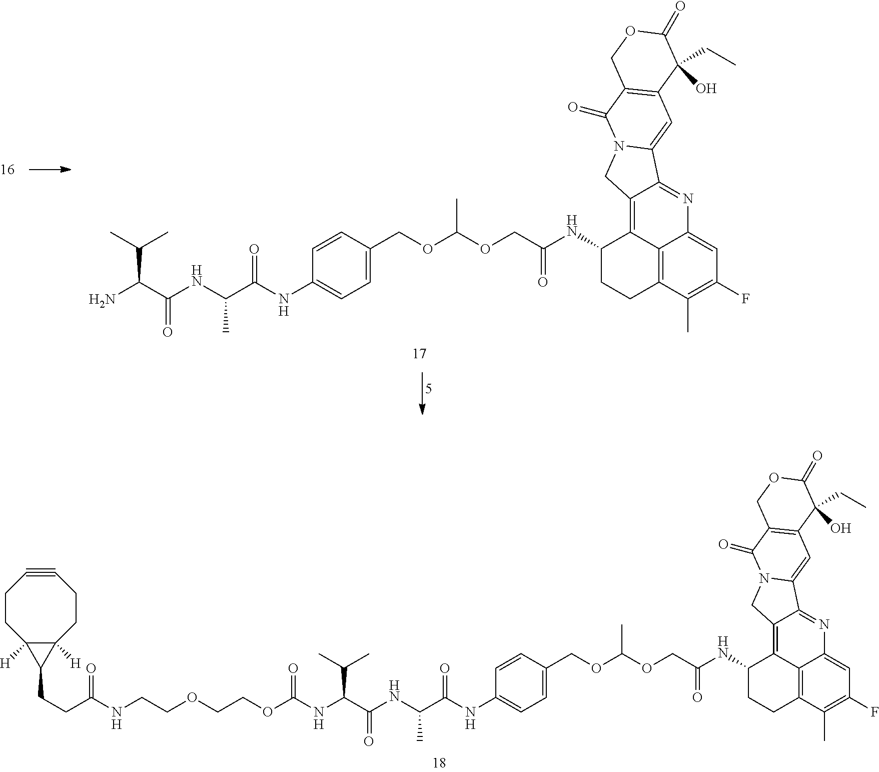 [SMILES: CC[C@@]1(O)C(=O)OCc2c1cc1n(c2=O)Cc2c-1nc1cc(F)c(C)c3c1c2[C@@H](NC(=O)COC(C)OCc1ccc(NC(=O)[C@H](C)NC(=O)[C@@H](N)C(C)C)cc1)CC3 ; CC[C@@]1(O)C(=O)OCc2c1cc1n(c2=O)Cc2c-1nc1cc(F)c(C)c3c1c2[C@@H](NC(=O)COC(C)OCc1ccc(NC(=O)[C@H](C)NC(=O)[C@@H](NC(=O)OCCOCCNC(=O)CC[C@@H]2[C@@H]4CCC#CCC[C@@H]42)C(C)C)cc1)CC3]

To a solution of 16 (2.2 mg, 2.1 μmol) in DMF (0.42 mL) was added piperidine (2.2μ, 1.9 mg, 22 μmol). The mixture was left standing for 1 h, diluted with DCM (2 mL) and purified with silica gel chromatography (DCM→20% MeOH in DCM). The desired product 17 was obtained as a white film (1.2 mg, 1.5 μmol, 71%). LCMS (ESI+) calculated for C₄₃H₅₀FN₆O₉⁺ (M+H⁺) 813.36. found 813.36.

To a solution of 5 (1.2 mg, 1.5 μmol) in DMF (0.3 mL) was added a solution of 17 (1.3 mg, 3.0 μmol) in DMF (15 μL) and Et₃N (1.0 μL, 0.76 mg). The mixture was left standing for 18 h, diluted with DCM (2 mL) and purified by silica gel chromatography (DCM→10% MeOH in DCM). The desired product 18 was obtained as a colourless film (2.1 mg, 1.9 μmol, quant.). LCMS (ESI+) calculated for C₅₉H₇₁FN₇O₁₄+ (M+H⁺) 1120.50. found 1120.50.

### Example 9. Synthesis of Compounds 21 and 22

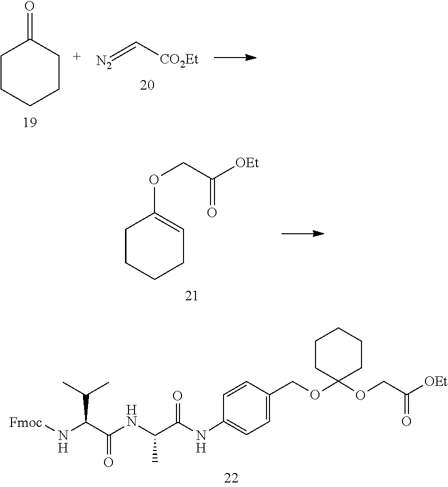

The following procedure was executed according to WO2010/009124 and Landgrebe et al, J. Org. Chem. 1978, 43, 1244-1245, incorporated by reference.

A mixture of cyclohexanone (19) (8.5 mL, 8.0 g, 82 mmol) and CuCl (17 mg, 0.17 mmol) was heated to 90° C. A solution of ethyl diazoacetate (20) (1.8 mL, 2.0 g, 17.5 mmol) in cyclohexanone (4.0 mL, 3.8 g, 39 mmol) was added and the resulting mixture was stirred at 90° C. until nitrogen formation stopped (20 min). The mixture was allowed to come to r.t. and concentrated in vacuo at 65° C. The residue was then purified by silica gel chromatography (toluene→10% Et₂O in toluene). The desired product 21 was obtained as a colourless liquid (0.94 g, 5.1 mmol, 29%). ¹H NMR (400 MHz, CDCl₃) δ (ppm) 4.57-4.52 (m, 1H), 4.30 (s, 2H), (4.24, J=7.3 Hz, 2H), 2.17-2.11 (m, 2H), 2.07-2.00 (m, 2H), 1.72-1.63 (m, 2H), 1.57-1.49 (m, 2H), 1.29 (t, J=7.2 Hz, 3H).

Under an atmosphere of nitro in a flame dried flask, to a solution of Fmoc-va-PAB-OH (1) (0.15 g, 0.29 mmol) in anhydrous DMF (4 mL) was added activated molsieves (53 mg). The mixture was stirred for 30 min and 4-toluenesulfonic acid monohydrate (18 mg, 0.096 mmol) was added. The mixture was stirred for 25 min and 21 (0.27 g, 1.4 mmol) was added. The mixture was stirred for 3 h and filtered. The residue was rinsed with anhydrous DMF, diluted with DCM and purified by silica gel chromatography (30%→70% EtOAc in heptane). The desired product 22 was obtained as a white solid (30 mg, 4.3 μmol, 15%). LCMS (ESI+) calculated for C₄₀H₄₉N₃NaO₈⁺ (M+Na⁺) 722.34. found 722.41.

### Example 10. Synthesis of Compounds 23 and 24

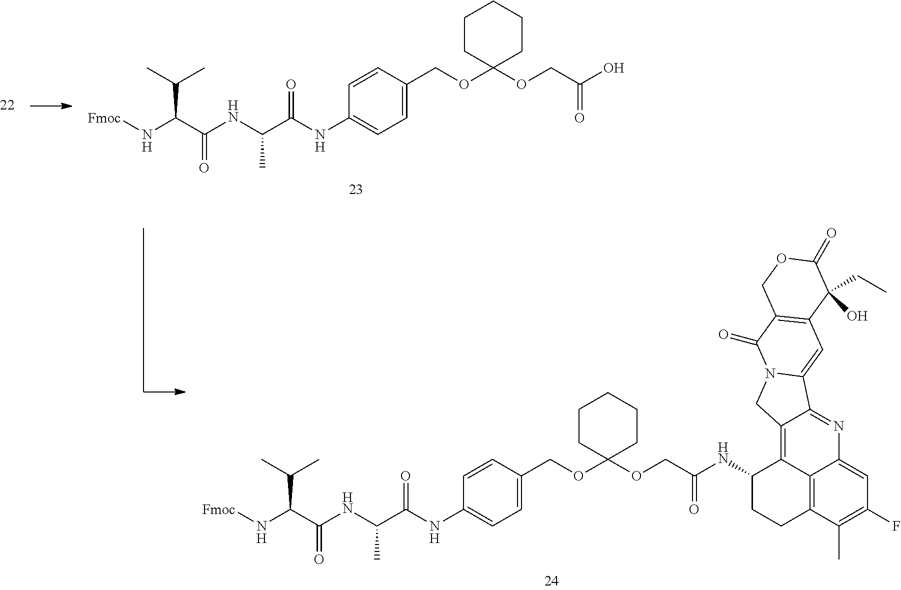

To a solution of 22 (30 mg, 43 μmol) in 1,2-dichloroethane (2 mL) was added Me₃SnOH (39 mg, 0.21 mmol). The mixture was heated to 80° C., stirred for 5 h, allowed to come to rt, diluted with DCM (5 mL) and purified by silica gel chromatography (DCM→25% MeOH in DCM), which afforded 16 mg (24 μmol, 56%) of the desired product 23. LCMS (ESI+) calculated for C₃₈H₄₅N₃NaO₈⁺ (M+Na⁺) 694.31. found 694.44.

To a suspension of exatecan mesylate (9) (1.5 mg, 2.8 μmol) in DMF (0.15 mL) were added a solution of N-hydroxysuccinimide (0.32 mg, 2.8 μmol) in DMF (13 μL), a solution of 23 (2.0 mg, 2.8 μmol) in DMF (38 μL), a solution of Dipea (0.32 mg, 0.43 μL, 2.8 μmol) in DMF (4.9 μL) and a solution of N,N′-dicyclohexylcarbodiimide (0.74 mg, 3.6 μmol) in DMF (28 μL). The mixture was placed on a tube roller for 10 min and then left standing for 1 h. The mixture was concentrated and taken up in DMF (50 μL). A solution of Dipea (0.32 mg, 0.43 μL, 2.8 μmol) in DMF (4.9 μL) was added. The mixture was left standing for 30 min and a solution of N-hydroxysuccinimide (0.32 mg, 2.8 μmol) in DMF (13 μL), a solution of 23 (2.0 mg, 2.8 μmol) in DMF (38 μL) and a solution of N,N′-dicyclohexylcarbodiimide (0.74 mg, 3.6 μmol) in DMF (28 μL) were added. The mixture was left standing for 2 d and purified by silica gel chromatography (DCM→10% MeOH in DCM). The desired product 24 was obtained as a colourless film (3.6 mg, 3.3 μmol, quant.) LCMS (ESI+) calculated for C₆₂H₆₆FN₆O₁₁⁺ (M+Na+) 1089.48. found 1089.56.

### Example 11. Synthesis of Compounds 25 and 26

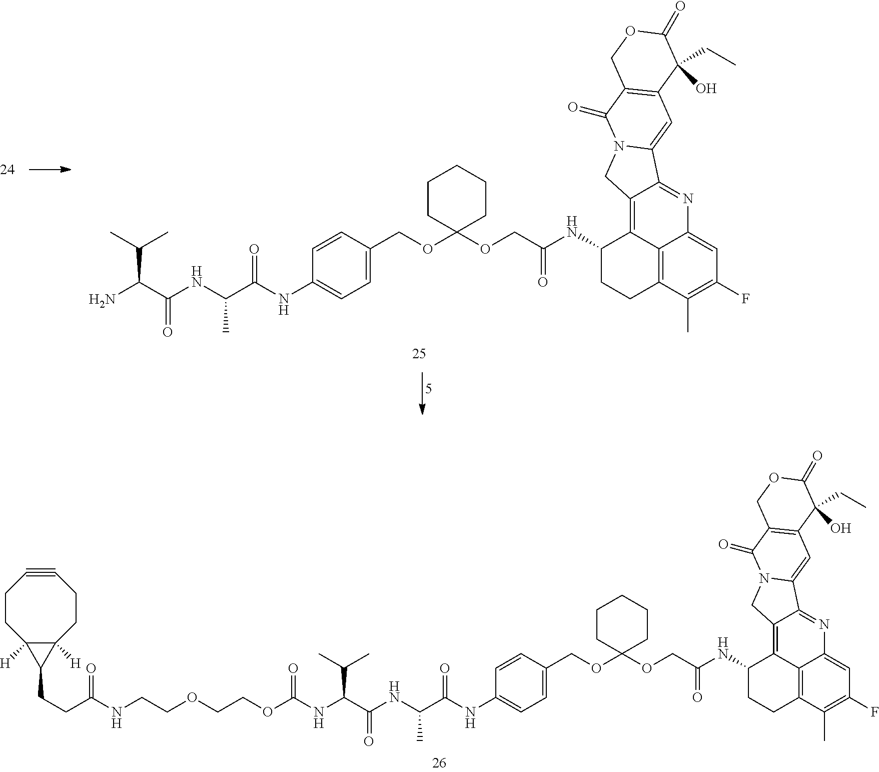 [SMILES: CC[C@@]1(O)C(=O)OCc2c1cc1n(c2=O)Cc2c-1nc1cc(F)c(C)c3c1c2[C@@H](NC(=O)COC1(OCc2ccc(NC(=O)[C@H](C)NC(=O)[C@@H](N)C(C)C)cc2)CCCCC1)CC3 ; CC[C@@]1(O)C(=O)OCc2c1cc1n(c2=O)Cc2c-1nc1cc(F)c(C)c3c1c2[C@@H](NC(=O)COC1(OCc2ccc(NC(=O)[C@H](C)NC(=O)[C@@H](NC(=O)OCCOCCNC(=O)CC[C@@H]4[C@@H]5CCC#CCC[C@@H]54)C(C)C)cc2)CCCCC1)CC3]

To a solution of 24 (3.6 mg, 3.3 μmol) in DMF (0.33 mL) was added piperidine (3.5 μL, 3.0 mg, 35 μmol). The mixture was left standing for 50 min, diluted with DCM (2 mL) and purified by silica gel chromatography (DCM→25% MeOH in DCM), which afforded 3.2 mg (3.7 μmol, quant.) of the desired compound 25. LCMS (ESI+) calculated for C₄₇H₅₆FN₆O₉⁺ (M+H⁺) 867.41. found 867.54.

To a solution of 25 (3.2 mg, 3.7 μmol) in DMF (0.37 mL) were added a solution of 5 (3.3 mg, 7.4 μmol) in DMF (39 μL) and Et₃N (2.6 μL, 1.9 mg, 18.5 μmol). The mixture was left over night, diluted with DCM (2 mL) and purified by silica gel chromatography (DCM→10% MeOH in DCM). The desired product 26 was obtained as a colourless film (2.1 mg, 1.8 μmol, 49%). LCMS (ESI+) calculated for C₆₃H₇₇FN₇O₁₄⁺ (M+H⁺) 1174.55. found 1174.68.

### Example 12. Results of Papain Digestion Experiments Including Negative Controls

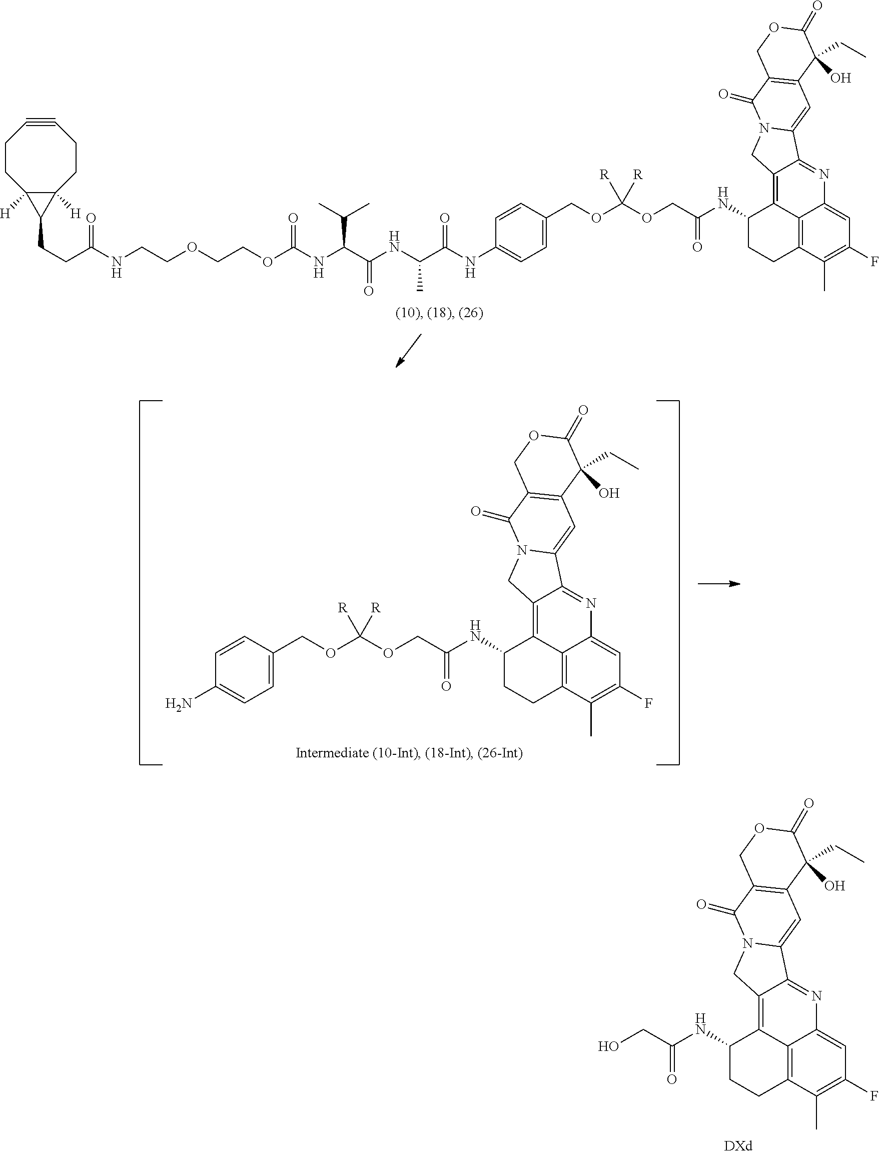

To an Eppendorf vial containing histidine acetate buffer (50 μL, 20 mM, pH 6) were added a solution of 10 in DMF (3 μL, 10 mM) and a Papain solution in histidine acetate buffer (0.5 mg/mL, 20 mM buffer, pH 6). After incubation for 19 h at 37° C., the reaction mixture was analysed by UPLC-MS which showed partial conversion of 10 into 10-Int while only a very small amount of material was transformed into DXd.

To an Eppendorf vial containing histidine acetate buffer (110 μL, 20 mM, pH 6) was added a solution of 10 in DMF (3 μL, 10 mM). After incubation for 18 h at 37° C., the reaction mixture was analysed by UPLC-MS which showed no conversion of 10 into 10-Int and DXd.

To an Eppendorf vial containing histidine acetate buffer (50 μL, 20 mM, pH 6) were added a solution of 18 in DMF (3 μL, 10 mM) and a Papain solution in histidine acetate buffer (0.5 mg/mL, 20 mM buffer, pH 6). After incubation for 19 h at 37° C., the reaction mixture was analysed by UPLC-MS which showed partial conversion of 18 into 18-Int and DXd.

To an Eppendorf vial containing histidine acetate buffer (110 μL, 20 mM, pH 6) was added a solution of 18 in DMF (3 μL, 10 mM). After incubation for 18 h at 37° C., the reaction mixture was analysed by UPLC-MS which showed no conversion of 18 into 18-Int and DXd.

To an Eppendorf vial containing histidine acetate buffer (50 μL, 20 mM, pH 6) were added a solution of 26 in DMF (3 μL, 10 mM) and a Papain solution in histidine acetate buffer (0.5 mg/mL, 20 mM buffer, pH 6). After incubation for 67 h at 37° C., the reaction mixture was analysed by UPLC-MS which showed partial conversion of 26 into DXd. A very small amount of the intermediate 26-Int was not observed.

To an Eppendorf vial containing histidine acetate buffer (110 μL, 20 mM, pH 6) was added a solution of 26 in DMF (3 μL, 10 mM). After incubation for 67 h at 37° C., the reaction mixture was analysed by UPLC-MS which showed partial conversion of 26 into DXd. The intermediate 26-Int was not observed.

To a 1:1 mixture of PBS (pH 7.4) and DMF (199 μL) was added a 10 mM solution of 10 in DMF (1 μL). The mixture was incubated overnight and analyzed by UPLC-MS. Intermediate 10-Int and DXd were not observed.

To a 1:1 mixture of PBS (pH 7.4) and DMF (199 μL) was added a 10 mM solution of 18 in DMF (1 μL). The mixture was incubated overnight and analysed by UPLC-MS. Intermediate 18-Int and DXd were not observed.

To a 1:1 mixture of PBS (pH 7.4) and DMF (199 μL) was added a 10 mM solution of 26 in DMF (1 μL). The mixture was incubated overnight and analysed by UPLC-MS. Intermediate 26-Int and DXd were not observed.

The results of the digestion experiments are summarized in the table below:

| Starting | | | Formation of | Formation | Ratio |
|---|---|---|---|---|---|
| material | pH | Papain | Intermediate | of DXd | DXd / s.m.^{#} |
| 10 | 6 | YES | + | + / − | N.D.* |
| | | NO | − | − | 0 |
| | 7.4 | NO | − | − | 0 |
| 18 | 6 | YES | + | + | N.D.* |
| | | NO | − | − | 0 |
| | 7.4 | NO | − | − | 0 |
| 26 | 6 | YES | − | + | 65:35 |
| | | NO | − | + / − | 3:97 |
| | 7.4 | NO | − | − | 0 |

* Intermediate and DXd are eluting at the same retention time
^{#}Based on peak area (UV 254 nm)

### Example 13. Preparation of ADCs

ADCs were prepared based on enzymatic remodeling of trastuzumab with 6-azidoGalNAc according to WO 2017/137457, example 6, WO 2016/170186, example 9 and WO 2014/065661, examples 20-28. ADCs were prepared by conjugation of bis(6-azidoGalNAc)-trastuzumab with BCN-linker-drugs 1-4 below.

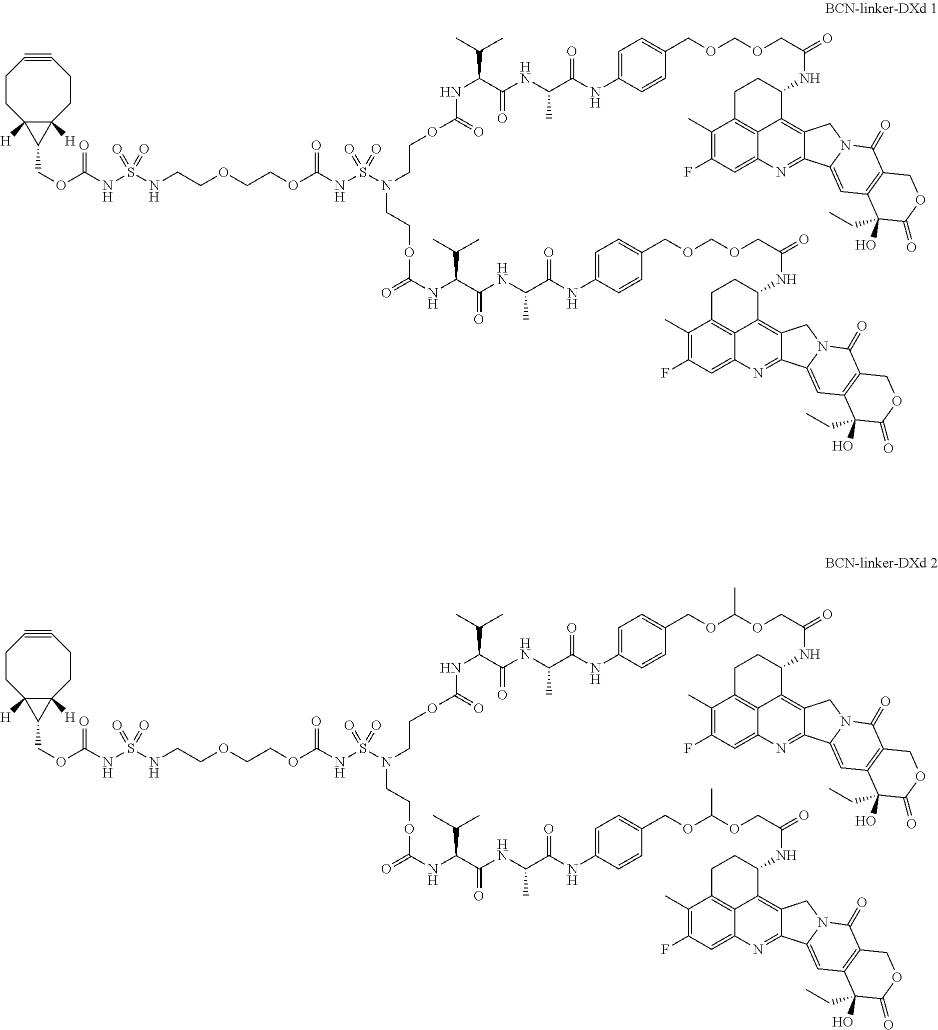

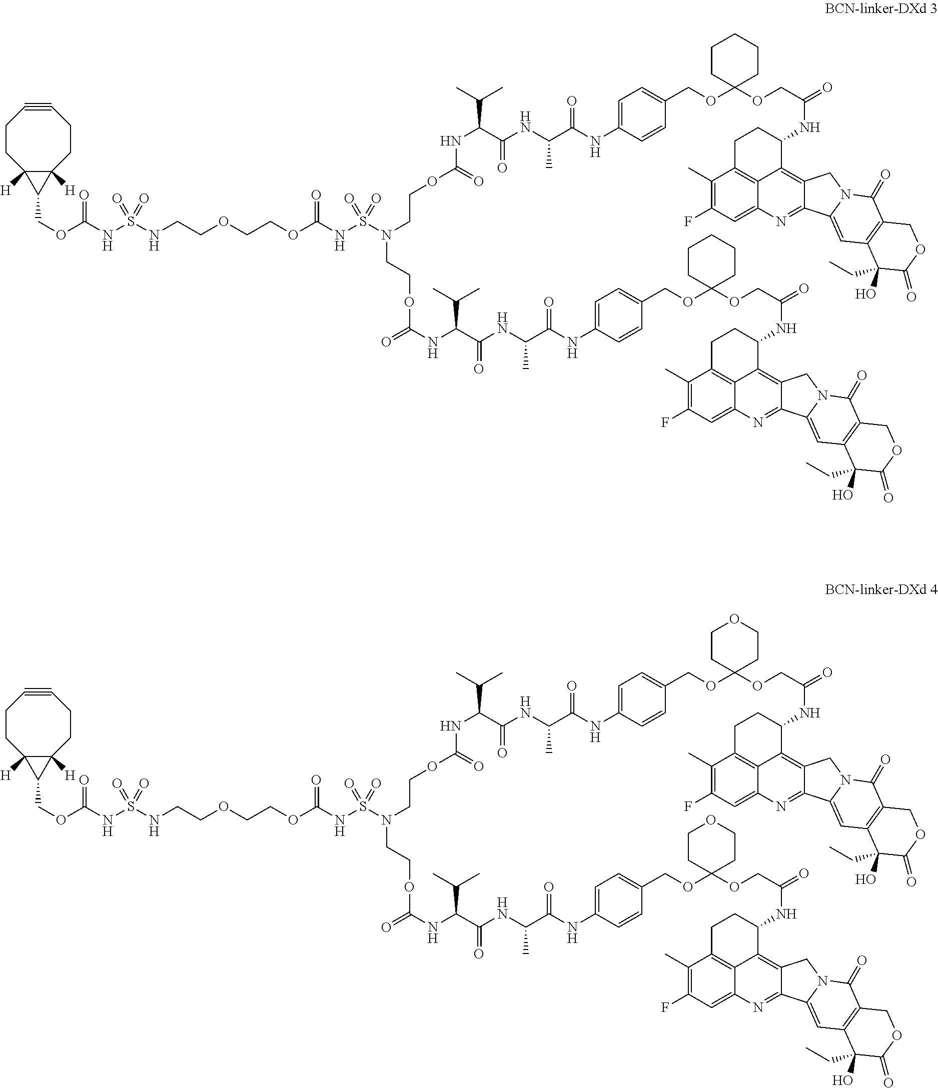

### Example 14. In Vitro Potency Determination of ADCs

In vitro potency of the ADCs based on trastuzumab and BCN-linker-DXd 1-4, as prepared in Example 13, was evaluated according to van Geel et al., Bioconj. Chem. 2015, 26, 2233-2242 using cell lines N₈₇, SK-BR-3 and BT-474. Efficient cell killing was observed for all four ADCs.

1. A compound according to structure (1): 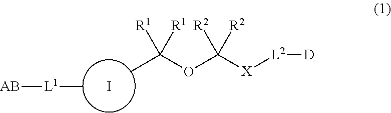
or a salt thereof, wherein
AB is an antibody or a reactive moiety capable of reacting with a functional group on an antibody;
L¹ is a linker;
moiety I is according to formula (2a), (2b), (2c) or (2d): 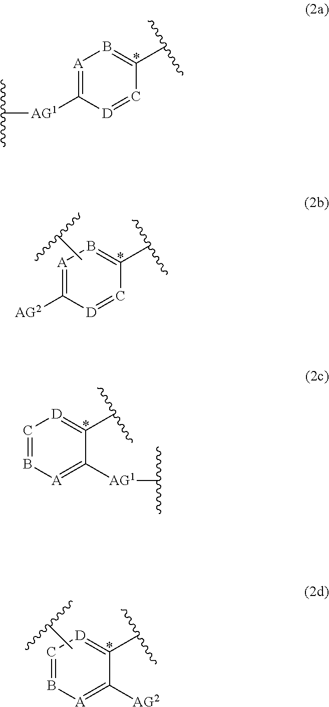
wherein
* indicates the carbon atom connected to C(R¹)₂;
each of A, B, C and D is independently selected from N and CR⁵, wherein R⁵ is H, optionally substituted alkyl, alkoxy, sulfonate, alkylamino or halogen wherein the alkyl may be interrupted by one or more heteroatoms, or in case of formula (2b) and (2d) at least one of A, B, C and D is CR⁵, wherein R⁵ is L¹-AB;
AG¹ is an activating group selected from —S—S— and —NH-peptide-, wherein the peptide contains 1-10 amino acids;
AG² is an activating group selected from the group consisting of —S—S—R¹⁰ and —NH-peptide-R¹¹, —NO₂, —N₃, —OR⁴ and —OC(O)R⁸, wherein the peptide contains 1-10 amino acids, wherein R⁴ is selected from phosphate, sulfate and monosaccharide, R⁸ is a C₂-C₁₂ alkyl group, R¹⁰ is an optionally substituted alkyl or aryl group; and R¹¹ is the optionally protected end group of the peptide;
each R¹ is independently selected from the group consisting of hydrogen, optionally substituted alkyl, optionally substituted aryl, C(O)OR⁶ and C(O)N(R⁶)₂;
each R² is independently selected from the group consisting of hydrogen, optionally substituted alkyl and optionally substituted aryl, or both occurrences of R² are joined together in a cyclic structure;
X is selected from O and NR³, wherein R³ is selected from the group consisting of H, alkyl, aryl, —C(O)-alkyl, —C(O)-aryl, —C(O)—O-alkyl, —C(O)—O-aryl, —C(O)—N(R⁶)₂, —C(O)—N(R⁶)₂, —S(O₂)—O-alkyl, S(O₂)—O-aryl, —S(O₂)—N(R⁶)₂ and —S(O₂)—N(R⁶)₂;
each R⁶ is independently selected from the group consisting of hydrogen, optionally substituted alkyl and optionally substituted aryl, or both occurrences of R⁶ on the same nitrogen atom are joined into in a cyclic structure;
L² is —C(O)-L³-C(O)— or -L³-C(O)—, wherein L³ is optionally substituted alkylene and/or optionally substituted arylene, wherein the alkylene may optionally be interrupted with 1-3 heteroatoms selected from O, NR⁷ and S(O)_{y}, wherein y=0, 1 or 2;
D is a payload.

2. The compound according to claim 1, wherein X is O.

3. The compound according to claim 1, wherein X is NR³ and:
R³ is —C(O)-alkyl or —C(O)-aryl, and L² is -L³-C(O)—; or
R³ is H, alkyl or aryl, and L² is —C(O)-L³-C(O)—.

4. The compound according to claim 1, wherein L³ is —(CH₂)_{b}—, wherein b is an integer in the range of 1-10.

5. The compound according to claim 1, wherein moiety I is according to formula (2a), and AG¹ is —NH-peptide.

6. The compound according to claim 1, wherein moiety I is according to formula (2b), and one of A, B, C and D is CR⁵, wherein R⁵ is L¹-AB, and AG² is —NH-peptide-R¹¹.

7. The compound according claim 1, wherein one occurrence of R¹ is hydrogen and the other occurrence of R¹ is hydrogen or C(O)N(R⁶)₂.

8. The compound according claim 1, wherein one occurrence of R² is hydrogen and the other occurrence of R² is hydrogen or C₁-C₆ alkyl.

9. The compound according claim 1, wherein payload D in unconjugated form contains an amino group, which is covalently attached to the C(O) moiety of L².

10. A method of targeting a cell, comprising contacting the cell with a compound according to claim 1, wherein AB is an antibody that specifically targets the cell or a receptor expressed on the cell.

11. The method of providing a payload to a cell, comprising contacting the cell with a compound according to claim 1, wherein AB is an antibody that specifically targets the cell or a receptor expressed on the cell, wherein a payload having formula HX-L²-D is released near or in the cell.

12. The method of enhancing the bystander effect of an amino-containing payload by conversion into an hydroxyl-containing payload HO-L²-D, comprising contacting a cell with a compound according to claim 1, wherein X=O and AB is an antibody that specifically targets the cell or a receptor expressed on the cell, wherein a payload having formula HO-L²-D is released near or in the cell.

13. The method according to claim 10 wherein the cell is present in a subject or a sample taken from a subject.

14. The method according to claim 10 wherein the environment near or in the cell is below physiological pH in the range of 4.5-6.8.

15. A compound having a structure selected from the group consisting of (5a)-(5i): 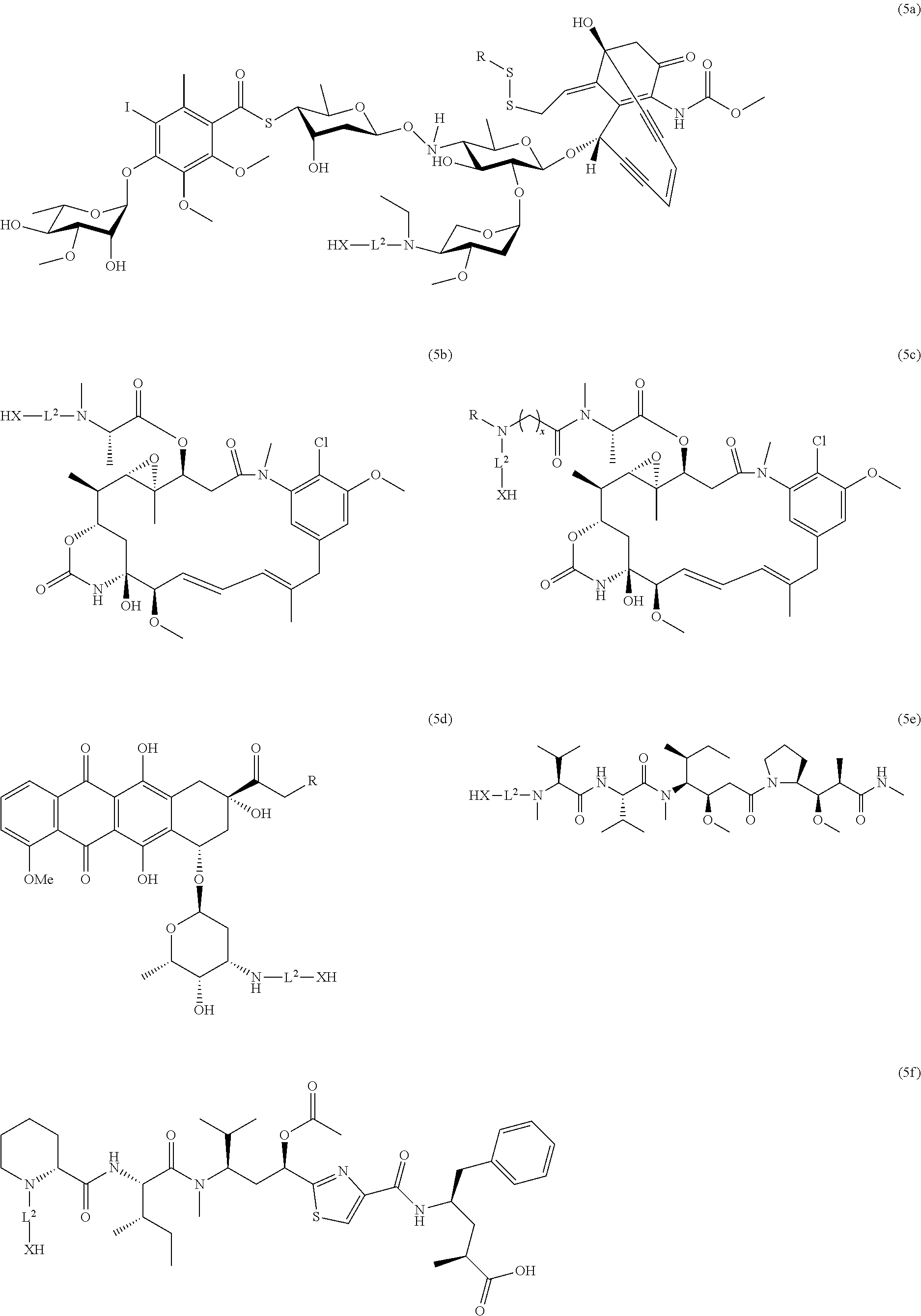 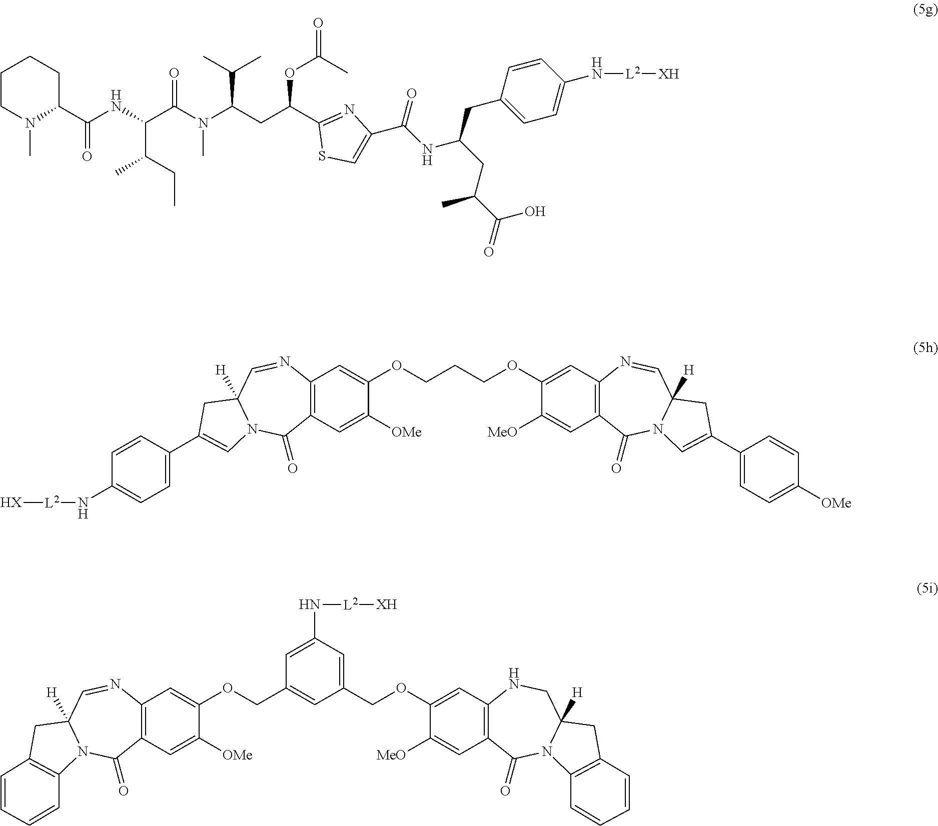
or a salt thereof, wherein:
X is selected from O and NR³, wherein R³ is selected from the group consisting of H, alkyl, aryl, —C(O)-alkyl, —C(O)-aryl, —C(O)—O-alkyl, —C(O)—O-aryl, —C(O)—N(R⁶)₂, —C(O)—N(R⁶)₂, —S(O₂)—O-alkyl, S(O₂)—O-aryl, —S(O₂)—N(R⁶)₂ and —S(O₂)—N(R⁶)₂;
L² is —C(O)-L³-C(O)— or -L³-C(O)—, wherein L³ is optionally substituted alkylene and/or optionally substituted arylene, wherein the alkylene or arylene is optionally substituted and the alkylene may optionally be interrupted with 1-3 heteroatoms selected from O and NR⁷, wherein R⁷ is selected from H and C₁-C₄ alkyl,
and wherein:
in structure (5a), R is —S(C₁-C₁₀ alkyl) or —C(R¹⁶)₂R¹⁷, wherein each R¹⁶ is independently selected from H or optionally substituted C₁-C₆ alkyl and R¹⁷ is selected from H, C₁-C₁₂ alkyl, -L⁶-OR¹⁸, (CH₂)ₛO-L⁶-OR¹⁸ or (CH₂)ₛC(O)NR¹⁹-L⁶-OR¹⁸, wherein L⁶ is a polar linker having 1-100 optionally substituted backbone atoms selected from C, N, O and S, R¹⁸ is H or methyl, s=1, 2 or 3, and R¹⁹ is selected from H and -L⁶-OR¹⁸;
in structure (5c), R is H or C₁-C₁₂ alkyl and x is an integer in the range 1-10;
in structure (5d), R is H or OH; and
in structure (5e), R is CH(Me)-CH(OH)-Ph.

16. The compound according to claim 15, the compound is according to structure (5a), (5b) or (5c).

17. The compound according to claim 15, wherein X=O and L³=para-phenylene, meta-phenylene, ortho-phenylene, C₁-C₆ alkylene, para-Ph-O—[C₁-C₆ alkylene], para-Ph-CH₂—O—[C₁-C₆ alkylene] and para-Ph-CH₂—NR⁷—[C₁-C₆ alkylene], which may optionally be substituted, preferably wherein L³=CH₂.

18. The compound according to claim 17, wherein L³ is CH₂.

19. The compound according to claim 1, wherein moiety I is according to formula (2a) or (2c), wherein AG¹ is —NH-peptide-, X is O and L³ is —(CH₂)_{b}—, wherein b is an integer in the range of 1-10.

20. The compound according to claim 1, wherein payload D in unconjugated form contains an amino group, which is covalently attached to the C(O) moiety of L².

21. The compound according to claim 1, wherein payload D is selected from exatecan, calicheamicin, maytansine, anthracyclin, aurastatin and PBD.
